SEQUENCE LISTING (3 sequences)

SEQ ID NO: 1 - protein, 20 aa, Artificial Sequence
  synthetic sequence
  MOD_RES (1)..(1): biotinylated Asp
DRPPPPVLFPGQPFGQPPLG

SEQ ID NO: 2 - protein, 20 aa, Artificial Sequence
  synthetic sequence
PPGLVQDGFPFPQPGPPPRL

SEQ ID NO: 3 - protein, 20 aa, Artificial Sequence
  synthetic sequence
DRPPPPVLFPGQPFGQPPLG

(19) United States
(12) Patent Application Publication
(10) Pub. No.: US 2021/0115061 A1
(43) Pub. Date: Apr. 22, 2021

(54) Small-Molecule HIV-1 Capsid Protein Inhibitors and Methods Using Same

(71) Applicant: DREXEL UNIVERSITY, Philadelphia, PA (US)

(72) Inventor: Simon COCKLIN, Philadelphia, PA (US)

(21) Appl. No.: 17/254,476

(22) PCT Filed: Jun. 21, 2019
(86) PCT No.: PCT/US2019/038516

§ 371 Date: Dec. 21, 2020

Related U.S. Application Data
(60) Provisional application No. 62/688,055, filed on Jun. 21, 2018.

(51) Int. Cl. C07D 495/04; C07D 231/56

(52) U.S. Cl. CPC C07D 495/04; A61K 45/06; C07D 231/56

(57) ABSTRACT

The present invention provides a method of treating, ameliorating, and/or preventing HIV-I infection in a subject, comprising the step of administering to the subject one or more of the compounds useful within the invention.

### CROSS-REFERENCE TO RELATED APPLICATION

This application claims priority under 35 U.S.C. § 119(e) to U.S. Provisional Patent Application Ser. No. 62/688,055 entitled “SMALL-MOLECULE HIV-1 CAPSID PROTEIN INHIBITORS AND METHODS USING SAME,” filed Jun. 21, 2018, the disclosure of which is incorporated herein by reference in its entirety.

### STATEMENT REGARDING FEDERALLY SPONSORED RESEARCH

This invention was made with government support under grant number AI118415-01A1 awarded by the National Institutes of Health. The government has certain rights in this invention.

### BACKGROUND OF THE INVENTION

Human immunodeficiency virus type 1 (HIV-1), the major causative agent of acquired immunodeficiency syndrome (AIDS), is a retrovirus of the genus lentivirinae. Retroviruses are small enveloped viruses that contain a diploid RNA genome. Each HIV-1 viral particle is composed of three discrete layers. The external surface of the virus is comprised of a lipid bilayer that is derived from the infected host cell. Embedded within this membrane are the viral envelope glycoproteins. The viral glycoproteins are organized on the virion surface as trimeric spikes, composed of three gp120 molecules non-covalently linked to three gp41 molecules, and function to mediate the entry of HIV-1 into susceptible cells. Below the lipid bilayer is a layer formed of the N-terminal region of the Gag polyprotein, known as the matrix (MA) protein. The third layer of the viral particle serves to protect the viral genome and replicative enzymes of HIV-1. This layer is a shell consisting of assembled mature capsid (CA) protein.

The HIV-1 CA protein performs essential roles both early and late in the life cycle of HIV: one structural, in which it forms a protein shell that shields both the viral genome and the replicative enzymes of HIV-1, and the other regulatory, in which the precise temporal disassembly of this shell coordinates post-entry events such as reverse transcription.

The HIV-1 CA protein is initially translated as the central region of the Gag polyprotein, where it functions in viral assembly and in packaging the cellular protein prolyl isomerase, cyclophilin A (CypA). As the virus buds, Gag is processed by the viral protease to produce three discrete new proteins—MA protein, CA protein, and nucleocapsid (NC)—as well as several smaller spacer peptides. After HIV-1 CA protein has been liberated by proteolytic processing, it rearranges into the conical core structure that surrounds the viral genome at the center of the mature virus.

The HIV-1 capsid shell is composed of about 250 CA protein hexamers and 12 CA protein pentamers, comprising about 1,500 monomeric CA proteins in all. The multimers interact non-covalently to form the shell's curved surface. CA protein itself is composed of two domains: the N-terminal domain (CA_{NTD}) and the C-terminal domain (CA_{CTD}). Both of these domains make critical inter- and intradomain interactions that are critical for the formation of the capsid shell. The structures of the individual domains, the NTD hexamer, the single CA protein, and the CA_{NTD} linked to MA have been determined. Both CA_{NTD} and CA_{CTD} are predominantly helical and are connected by a short flexible linker.

The CAN is composed of an N-terminal β-hairpin, seven α-helices, and an extended loop connecting helices 4 and 5 that binds CypA. CA protein residues 146 and 147 act as a flexible linker that connects the CA_{NTD} with the smaller CA_{CTD}, which is composed of four α-helices. The CTD dimerizes in solution and in the crystal, and contains an essential stretch of 20 amino acids (the major homology region) that is highly conserved in all retroviruses.

The structure of the CA protein hexamer reveals that six NTDs form the rigid core of hexameric CA protein, and six CTDs form the hexamer's much more flexible outer ring. Dimeric interactions between CTDs of neighboring hexamers hold the capsid together.

NTD-NTD interactions are responsible for the formation of the HIV-1 CA protein hexameric configuration. NTD-NTD interactions are mediated through helices 1, 2, and 3, which associate as an 18-helix bundle in the center of the hexamer. The interface is primarily stabilized by hydrophilic contacts (bridging water molecules, hydrogen bonds, and salt bridges). However, the interface contains a small hydrophobic core of residues (L20, P38, M39, A42, and T58).

Extensive mutagenesis of the NTD domain has been performed. In addition to the mutations that perturb normal particle assembly, specific mutations in the NTD that either destabilized or stabilized the structure of the CA protein hexamer had adverse affects on viral replication. Some residues of importance include the intersubunit stabilizing residues (E45, E128, and R132), the intersubunit destabilizing residues (R18, N21, P38, Q63, Q67, and L136), and the residues that when mutated reduce the rate of polymerization (A22, E28, and E29). Perhaps most interesting are two residues, M39 and A42, that when mutated completely prevent capsid assembly, as these may denote a potential “hotspot” for hexamerization. All of these types of mutations (stabilizing, destabilizing, and polymerization rate reducing) have a detrimental effect on the fitness of the virus. The inhibitory effects of mutations that modulate the stability of the capsid further highlight the need for a very delicate balance of favorable and unfavorable interactions within HIV-1 CA protein to allow assembly but also facilitate the uncoating process following infection.

There remains a need in the art to identify novel small-molecule inhibitors that bind to HIV-1 CA protein and interfere with one or more of its biological functions, leading to impairment of HIV-1 life cycle and infection. The present invention fulfills these needs.

### BRIEF SUMMARY OF THE INVENTION

In various embodiments, methods of identifying new small-molecule HIV-1 CA protein inhibitors are provided. The methods use in silico techniques to optimize the molecular structure of a starting compound, PF-74, into compounds that are predicted to be have better metabolic stability and good potency. Selected compounds are subsequently tested using in vitro assays and other experimental techniques. In some embodiments, the methods described herein were successfully used to identify potent HIV-1 CA protein inhibitors that have significantly improved ADME (absorption distribution metabolism elimination) and PK (pharmacokinetic) properties compared to the starting compound PF-74.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 shows the structure of compound PF-74.

FIG. 2 shows computed drug properties of compounds C1-C4 and pharmacokinetic properties for compound C4.

FIG. 3 shows compounds of Formula I having HYDE predicted affinities of less than 20 nM.

FIG. 4 shows the structures and computed ΔG (free energy of binding) and K_{D} values for compounds C11 and C12.

FIGS. 5A-5D show calculated and experimental properties of PF-74. FIG. 5A shows the structure of PF-74 and calculated solution-phase parameters. FIG. 5B shows sensorgrams depicting the interaction of PF-74 with sensor chip immobilized HIV-1_{NL4-3} CA hexamer. Blue lines depict actual binding data, black lines show fit to a 1:1 binding model. FIG. 5C shows potencies of PF-74 in the single round infection assay using Env pseudo-typed HIV-1 virus. IC₅₀, CC₅₀, and therapeutic indexes (TI=CC₅₀/IC₅₀) are also shown. FIG. 5D shows a metabolic stability evaluation of PF-74 in human liver microsomes.

FIGS. 6A-6D show calculated and experimental properties of CX03. FIG. 6A shows the structure of CX03 and calculated solution-phase parameters. FIG. 6B shows sensorgrams depicting the interaction of CX03 with sensor chip immobilized HIV-1_{NL4-3} CA hexamer. Blue lines depict actual binding data, black lines show fit to a 1:1 binding model. FIG. 6C shows potencies of CX03 in the single round infection assay using Env pseudo-typed HIV-1 virus. IC₅₀, CC₅₀, and therapeutic indexes (TI=CC₅₀/IC₅₀) are also shown. FIG. 6D shows a metabolic stability evaluation of CX03 in human liver microsomes.

FIGS. 7A-7D show calculated and experimental properties of C4. FIG. 7A shows the structure of C4 and calculated solution-phase parameters. FIG. 7B shows sensorgrams depicting the interaction of C4 with sensor chip immobilized HIV-1_{NL4-3} CA hexamer. Blue lines depict actual binding data, black lines show fit to a 1:1 binding model. FIG. 7C shows potencies of C4 in the single round infection assay using Env pseudo-typed HIV-1 virus. IC₅₀, CC₅₀, and therapeutic indexes (TI=CC₅₀/IC₅₀) are also shown. FIG. 7D shows a metabolic stability evaluation of C4 in human liver microsomes.

FIG. 8 illustrates important steps in the screening cascade to identify potent compounds.

FIG. 9 illustrates a computational workflow for designing improved compounds.

FIGS. 10A-10C show the interactions between PF-74 and the HIV-1 capsid. FIG. 10A shows the X-ray structure (PDB code 4qnb) of PF-74 complexed to fully assembled HIV-1. FIG. 10B illustrates key binding interactions between PF-74 and the HIV-1 capsid binding site. FIG. 10C illustrates a strategy to develop SAR (structure-activity relationships) and improve on the potency of PF-74.

FIG. 11 is a SDS-PAGE gel showing the IMAC purified monomeric forms of the CA proteins from HIV-1 reference strains. Lane 1. CDKTB48 (A1); 2. 92UG037 (A2); 3. NL4-3 (B); 4. YU2 (B); 5. 92BR025 (C); 6. 94UG114.1 (D). Respective clades are indicated in parentheses.

FIG. 12 shows the kinetic and thermodynamic parameters corresponding to the binding of PF74, C4, C11, and C12 to HIV-1 CA hexamer protein.

FIG. 13 shows the comparative binding between enantiomers of PF-74, (R)-PF-74 and (S)-PF-74 with HIV-1 CA hexamer protein.

FIG. 14 shows the comparative binding between enantiomers of C4, (R)-C4 and (S)-PF-C4 with HIV-1 CA hexamer protein.

FIG. 15 shows the comparative binding between enantiomers of C11, (R)-C11 and (S)-C11 with HIV-1 CA hexamer protein.

FIG. 16 shows the comparative binding between enantiomers of C12, (R)-C12 and (S)-C12 with HIV-1 CA hexamer protein.

FIG. 17 shows the binding of C13 to HIV-1 CA hexamer protein.

### DETAILED DESCRIPTION OF THE INVENTION

The present invention relates to the discovery that certain compounds are useful to treat or prevent HIV-1 viral infection in a vertebrate cell. In certain embodiments, these compounds bind to HIV-1 CA protein and act as antagonists or agonists of HIV-1 capsid hexamerization. In certain embodiments, these compounds inhibit or disturb one or more of the biological functions of the HIV-1 CA protein and therefore compromise the virus life cycle.

In one aspect, the invention provides a method of treating or preventing HIV-1 viral infection in a subject. The method comprises the step of administering the subject with a 20 therapeutically effective amount of a pharmaceutical composition comprising a compound that disrupts one or more of the biological functions of the HIV-1 CA protein. In one embodiment, the subject is human.

In another aspect, this application discloses novel HIV-1 inhibitors that target a highly conserved functional pocket present between CA protomers in the assembled hexamer. The compounds identified within this application can be used to probe the biological functions of HIV-1 CA protein, such as uncoating and assembly, and represent a new class of anti-HIV agents.

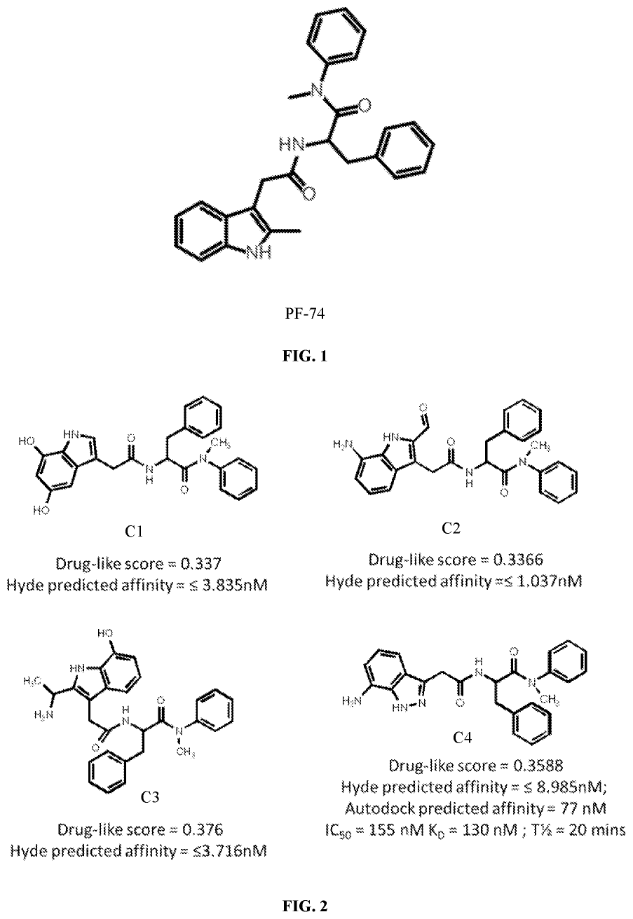

Because of the emergence of drug-resistant strains and the cumulative toxicities associated with current therapies, demand remains for new inhibitors of HIV-1 replication. An attractive target for such new inhibitors is the HIV-1 CA protein and specifically an inter-protomer pocket that serves as a binding site for host cell proteins. A small molecule, PF-74 (FIG. 1), has been identified which interacts in this area, demonstrating that it is amenable to small molecule targeting. However, this compound is incredibly metabolically labile, which severely limits its usefulness as a lead compound. Therefore, in this study, we design CA-targeted inhibitors that interact with the inter-protomer pocket and that have improved ADME/PK properties, including metabolic stability, and improved potencies. To do this, we use an innovative integration of both established and new computer-aided drug design (CADD) techniques, ADME/PK prediction and analysis, interaction analysis using surface plasmon resonance (SPR), and in vitro antiviral potency testing. Compound C4 has an IC₅₀ approximately 2-fold better than PF-74, improved predicted drug-like properties over PF-74, over 7-fold increase in the therapeutic index (CC₅₀/IC₅₀), and 34-fold better metabolic stability than PF-74.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. “About” as used herein when referring to a measurable value such as an amount, a temporal duration, and the like, is meant to encompass variations of ±20% or 10%, more preferably ≅5%, even more preferably ±1%, and still more preferably ±0.1% from the specified value, as such variations are appropriate to perform the disclosed methods.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I): an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

As used herein, the term “polypeptide” refers to a polymer composed of amino acid residues, related naturally occurring structural variants, and synthetic non-naturally occurring analogs thereof linked via peptide bonds. Synthetic polypeptides may be synthesized, for example, using an automated polypeptide synthesizer. As used herein, the term “protein” typically refers to large polypeptides. As used herein, the term “peptide” typically refers to short polypeptides. Conventional notation is used herein to represent polypeptide sequences: the left-hand end of a polypeptide sequence is the amino-terminus, and the right-hand end of a polypeptide sequence is the carboxyl-terminus.

As used herein, the term “antiviral agent” means a composition of matter which, when delivered to a cell, is capable of preventing replication of a virus in the cell, preventing infection of the cell by a virus, or reversing a physiological effect of infection of the cell by a virus. Antiviral agents are well known and described in the literature. By way of example, AZT (zidovudine, RETROVIR®, GlaxoSmithKline, Middlesex, UK) is an antiviral agent that is thought to prevent replication of HIV in human cells.

As used herein, the term “treatment” or “treating” is defined as the application or administration of a therapeutic agent, i.e., a compound useful within the invention (alone or in combination with another pharmaceutical agent), to a subject, or application or administration of a therapeutic agent to an isolated tissue or cell line from a subject (e.g., for diagnosis or ex vivo applications), who has an HIV-1 infection, a symptom of an HIV-1 infection or the potential to acquire an HIV-1 infection, with the purpose to cure, heal, alleviate, relieve, alter, remedy, ameliorate, improve or affect the HIV-1 infection, the symptoms of the HIV-1 infection or the potential to acquire the HIV-1 infection. Such treatments may be specifically tailored or modified, based on knowledge obtained from the field of pharmacogenomics.

As used herein, the term “IC₅₀” is the half-maximal inhibitory concentration of a particular compound or agent.

As used herein, the term “CC₅₀” is the half-maximal cytotoxic concentration of a particular compound or agent.

As used herein, the term “therapeutic index” is defined as the CC₅₀/IC₅₀ ratio.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient” or “subject” refers to a human or a non-human animal. Non-human animals include, for example, livestock and pets, such as ovine, bovine, porcine, canine, feline and murine mammals. Preferably, the patient or subject is human.

As used herein, the terms “effective amount,” “pharmaceutically effective amount” and “therapeutically effective amount” refer to a non-toxic but sufficient amount of an agent to provide the desired biological result. That result can be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

As used herein, the term “instructional material” includes a publication, a recording, a diagram, or any other medium of expression that may be used to communicate the usefulness of the compounds useful within the invention. In some instances, the instructional material may be part of a kit useful for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the compounds useful within the invention or be shipped together with a container that contains the compounds. Alternatively, the instructional material may be shipped separately from the container with the intention that the recipient uses the instructional material and the compound cooperatively. For example, the instructional material is for use of a kit; instructions for use of the compound; or instructions for use of a formulation of the compound.

### Compositions

The composition of the invention comprises compounds that may be synthesized using techniques well-known in the art of organic synthesis.

In various embodiments, a compound of Formula I, or a pharmaceutically acceptable salt, solvate, or tautomer thereof has the structure:

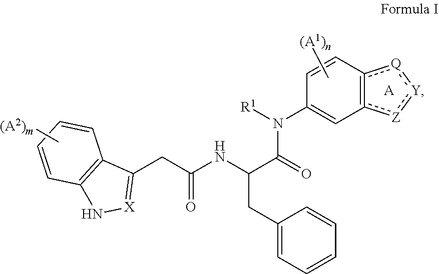

wherein

[symbol] is a covalent double bond or a covalent single bond;

ring A is optionally absent;

R¹ is hydrogen or a C₁₋₅ alkyl;

at each occurrence A¹ is independently selected from the group consisting of F, Cl, Br, I, OR, CN, NO₂, CF₃, OCF₃, R, N(R)₂, SR, SO₂F, SO₂R, SO₂N(R)₂, SO₃R, and (CH₂)₁₋₃OR;

at each occurrence A² is independently selected from the group consisting of F, Cl, Br, I, OR, CN, NO₂, CF₃, OCF₃, R, N(R)₂, SR SO₂F, SO₂R, SO₂N(R)₂, SO₃R, and (CH₂)₁₋₃OR;

X is N, C—C(═O)R, or C—R, wherein R is optionally substituted by 1 to 3 groups selected from the group consisting of NH₂ and OH;

Q, Y, and Z are each independently N, NH, or CH, wherein at least one of Q, Y, and Z is N or NH;

at each occurrence R is independently hydrogen or C₄ alkyl;

m is an integer from 0 to 5; and

n is an integer from 0 to 5.

In some embodiments, the compound has the structure of Formula Ia or Ib:

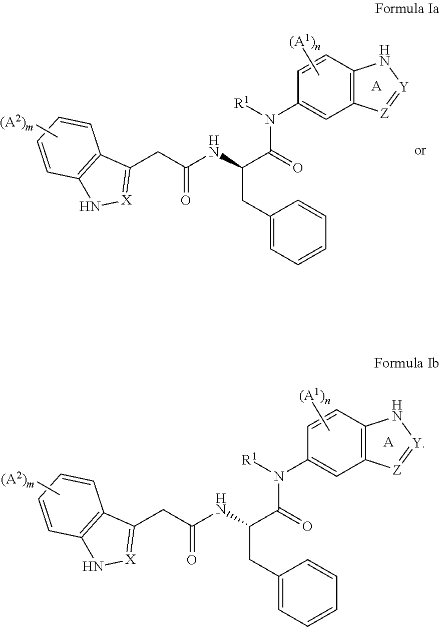

In various embodiments, the compound has the structure of Formula IIa or IIb:

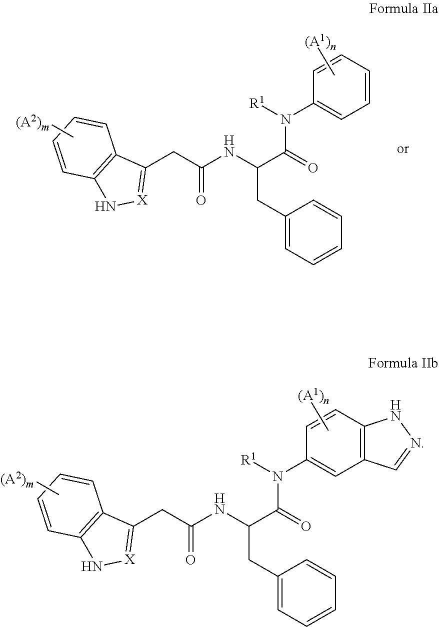

In some embodiments, X is N. In various embodiments, X is CH or C—CH₃. In various embodiments. R¹ is CH₃.

In one embodiment, the compound has the structure:

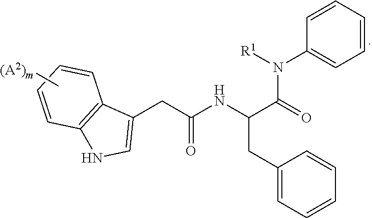

In another embodiment, the compound has the structure:

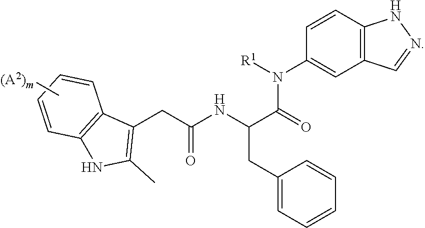

In some embodiments, the compound has the structure:

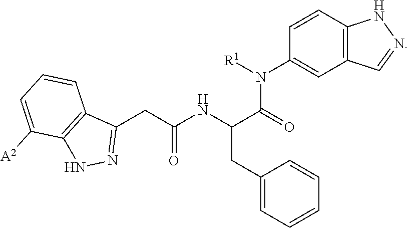

In various embodiments, m is 1. In some embodiments, A² is NH₂.
In various embodiments, the compound is selected from the group consisting of:

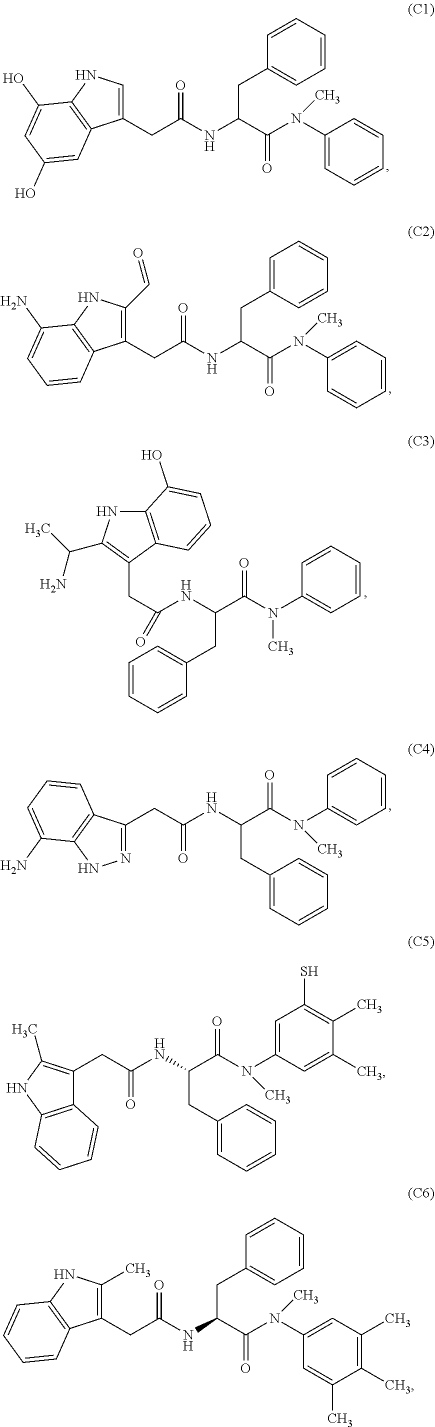

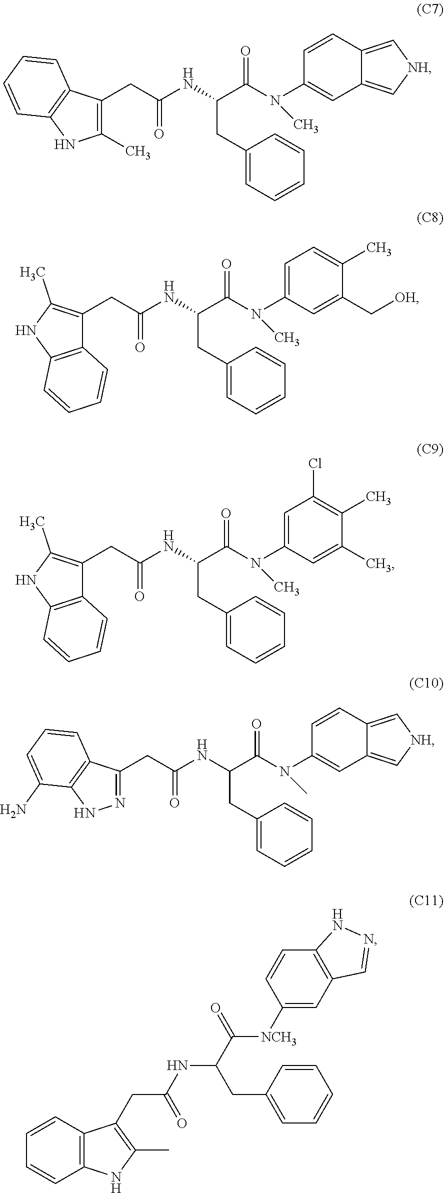 [SMILES: Cc1[nH]c2ccccc2c1CC(=O)N[C@@H](Cc1ccccc1)C(=O)N(C)c1ccc2c[nH]cc2c1 ; Cc1ccc(N(C)C(=O)[C@H](Cc2ccccc2)NC(=O)Cc2c(C)[nH]c3ccccc23)cc1CO ; Cc1cc(N(C)C(=O)[C@H](Cc2ccccc2)NC(=O)Cc2c(C)[nH]c3ccccc23)cc(Cl)c1C ; CN(C(=O)C(Cc1ccccc1)NC(=O)Cc1n[nH]c2c(N)cccc12)c1ccc2c[nH]cc2c1 ; Cc1[nH]c2ccccc2c1CC(=O)NC(Cc1ccccc1)C(=O)N(C)c1ccc2[nH]ncc2c1]

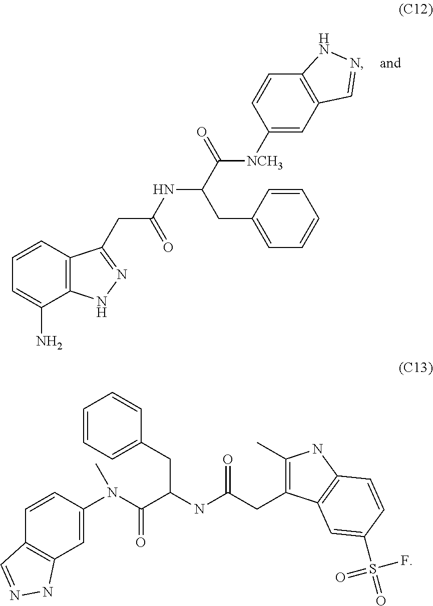 [SMILES: CN(C(=O)C(Cc1ccccc1)NC(=O)Cc1n[nH]c2c(N)cccc12)c1ccc2[nH]ncc2c1 ; Cc1[nH]c2ccc(S(=O)(=O)F)cc2c1CC(=O)NC(Cc1ccccc1)C(=O)N(C)c1ccc2cn[nH]c2c1]

In various embodiments, the compound is selected from the group consisting of:

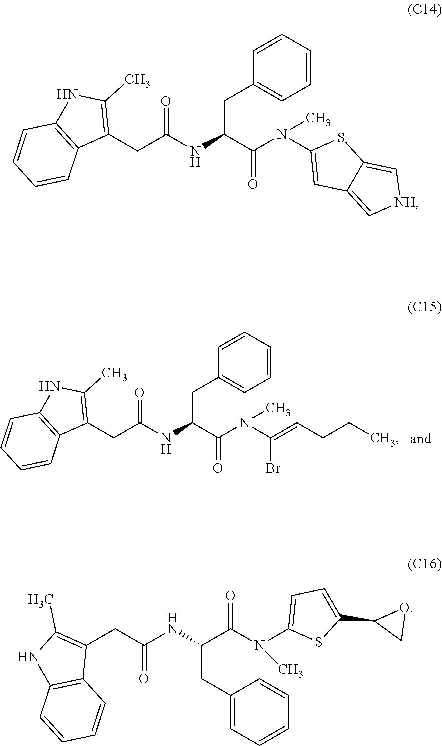 [SMILES: Cc1[nH]c2ccccc2c1CC(=O)N[C@@H](Cc1ccccc1)C(=O)N(C)c1cc2c[nH]cc2s1 ; CCC/C=C(\Br)N(C)C(=O)[C@H](Cc1ccccc1)NC(=O)Cc1c(C)[nH]c2ccccc12 ; Cc1[nH]c2ccccc2c1CC(=O)N[C@@H](Cc1ccccc1)C(=O)N(C)c1ccc([C@@H]2CO2)s1]

The HIV-1 CA performs essential roles both early and late in the life cycle of HIV. The capsid is initially translated as the central region of the Gag polyprotein. As the virus buds, Gag is processed by the viral protease to produce three discrete new proteins—matrix protein (MA), CA, and nucleocapsid (NC)—as well as several smaller spacer peptides. After the capsid has been liberated by proteolytic processing, it rearranges into the conical core structure that surrounds the viral genome at the center of the mature virus.

The HIV-1 capsid shell is composed of about 250 CA hexamers and 12 CA pentamers, comprising about 1500 monomeric CA proteins in total. The multimers interact noncovalently to form the shell's curved surface. CA itself is composed of two domains: the N-terminal domain (CA_{NTD}) and the C-terminal domain (CA_{CTD}).

To date, the CA protein structure and stability have been demonstrated to be critical for the processes of uncoating, reverse transcription, nuclear entry, selection of the sites of integration, and assembly. Moreover, it is also important for cloaking the DNA product from intracellular immune surveillance. To achieve these functions, HIV-1 CA interacts not only with itself but with host factors including TRIM5α, cleavage and polyadenylation specific factor 6 (CPSF6), nucleoporins 153 and 358 (NUP153, NUP358), MxB, and Cyclophilin A (CypA).

Drug-resistance and toxicity to antiretroviral drugs is a significant problem that drives the search for new inhibitors of HIV. Therefore, identifying new targets and developing therapeutic compounds remains a continuing research priority. Recent efforts by both academic and pharmaceutical researchers have focused on the HIV-1 capsid (CA) protein as a new therapeutic target. An inter-protomer pocket within the hexamer configuration of the CA, which is also a binding site for key host dependency factors, is the target of the very high potency compound GS-CA1 (Perrier, et al., 2017, J Antimicrob Chemother. 72(10):2954-2955) and the most widely studied CA inhibitor compound PF-74 (FIG. 1). Despite the fact that these compounds demonstrate that high-affinity compounds can be designed to this binding site, they, unfortunately, suffer from problems. GS-CA1 suffers from low solubility/bioavailability, and a greater than 28-step synthesis, and PF-74 is highly metabolically labile (Table 1).

TABLE 1
Metabolic clearance of testosterone, propranolol,
warfarin, and PF-74 in human liver microsomes.

| | | T_{1/2} | Clᵢₙₜ | Clₐₚₚ | Clₕ | Eₕ |
|---|---|---|---|---|---|---|
| Compound ID | k | (min) | (mL/min/mg) | (mL/min/kg) | (mL/min/kg) | (%) |
| Testosterone | 0.03852 | 18.0 | 0.0770 | 74.292 | 15.758 | 78.79 |
| Propranolol | 0.01887 | 36.7 | 0.0377 | 36.391 | 12.907 | 64.53 |
| Warfarin | −0.00509 | stable | / | / | / | / |
| PF-74* | 1.16637 | 0.59 | 2.3327 | 2249.427 | 19.824 | 99.12 |

Given the enormous potential of this inter-protomer pocket, compounds were designed that interact in this region, inhibit HIV-1 replication (preferentially at an early post-entry stage), but display much better ADME/PK properties and potencies in the clinically relevant range. Improved ADME/PK properties were achieved by using a multidisciplinary approach, combining computational methods, classical medicinal chemistry methods, biomolecular interaction analysis, antiviral potency determination, and in vitro ADME/PK assays.

The HIV-1 CA protein contains an inter-protomer pocket that serves as a binding site for host cell proteins. A small molecule, PF-74, interacts in this area, demonstrating that it is amenable to small molecule targeting. However, as illustrated in Table 1. PF-74 is metabolically labile, which severely limits its usefulness as a lead compound. PF-74 was used as a starting point to design and synthesize CA-targeted inhibitors that have improved metabolic stability and potencies by medicinal chemistry means. To accomplish this, an innovative integration of both established and new computer-aided drug design (CADD) techniques, metabolic stability prediction and analysis, interaction analysis using surface plasmon resonance (SPR), and in vitro antiviral potency testing was used.

Without limitation, the steps used to design and test HIV-1 CA protein binders with good ADME and PK properties, include:
i. the use of high-content pharmacophors based upon field points (field points are a condensed representation of the compound's shape, electrostatics, and hydrophobicity as calculated using the XED force field), in the identification, diversification and optimization of anti-HIV compounds targeting the HIV-1 CA protein;
ii. computational workflow that combines virtual bioisosteric replacements, HYdrogen bond and DEhydration (HYDE) energy scoring-based prediction of affinity, computational ADME/PK prediction, and re-docking of compounds to the CA hexamer, before synthesis and experimental testing;
iii. development of binding and competition assays using surface plasmon resonance that allows to prioritize compounds with favorable kinetic signatures (fast on, slow off) towards the hexameric configuration of the CA protein and demonstrate that the compounds bind within the inter-protomer binding pocket; and
iv. generation of novel reagents to assess potential cross-clade differences in affinity of the newly generated compounds. These include monomeric and hexameric forms of the CA protein from HIV-1 clade A1, A2, B, C, and D references strains.

In some embodiments, the steps i) through iv) led to the design compounds of Formula I described herein. In one embodiment, compound C4 was designed and tested. Compound C4 has an IC₅₀ approximately 2-fold better than PF-74, improved predicted drug-like properties over PF-74, over 7-fold increase in the therapeutic index (CC₅₀/IC₅₀), and 34-fold better metabolic stability than PF-74.

Multi-Step Computational Design of C4—a Compound with Improved Potency, Drug-Like Properties, Therapeutic Index, and Metabolic Stability Over PF-74

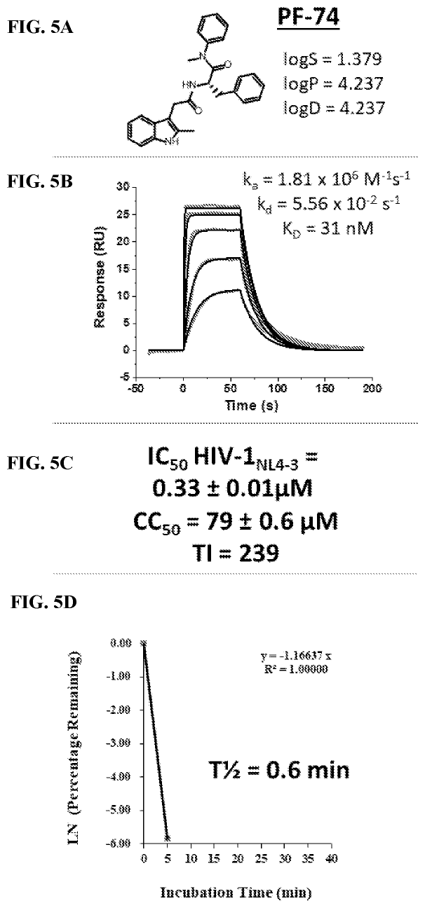

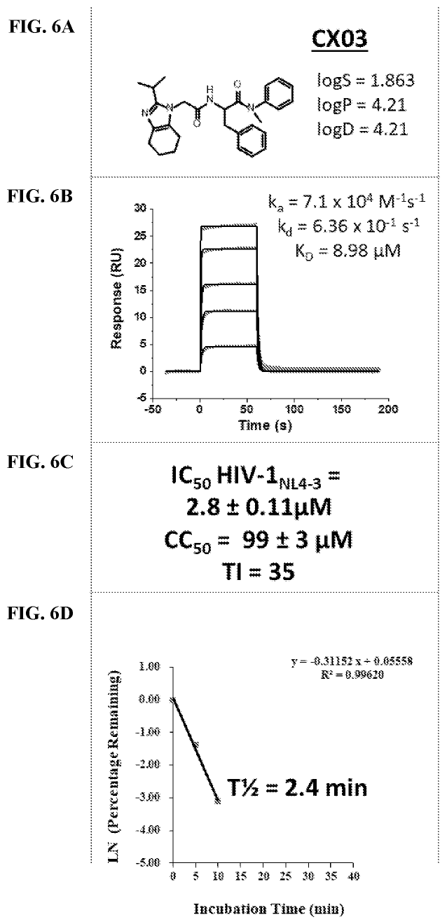

The structure of PF-74 bound to the native HIV-1 capsid hexamer structure (PDB ID 4XFZ) was imported into Spark Version 10.4 (Cresset*, Litlington, Cambridgeshire, UK; www dot cresset-group dot com/spark/) to derive a field-based high-content phamacophore. Two protomers, extracted from the native hexameric HIV-1 capsid protein structure, were added as excluded volume to discourage the selection of bioisosteric fragments that would clash with the protein within the inter-protomer pocket during the search. The methylindole group of PF-74 was chosen as the first area to study replacements. Spark searches a database, which includes fragments derived from multiple databases, to find non-classical bioisosteres that exhibit similar shape and electronic properties as the region of interest when placed in the context of the final molecule. The results of this search were analyzed and structures that displayed low 2D similarity, whilst retaining a sufficiently high BIF % value (a factor that indicates how good the replacement is in the context of the conformation of the entire molecule) were favored. Finally, the suggested molecules were analyzed for their predicted ADME properties (absorption, distribution, metabolism, and excretion) and compared to PF-74. The in silico prediction of drug-like metrics was achieved using StarDrop 6.4 (Optibrium, Ltd., Cambridge, UK). Following these analyses, compound CX03, containing a 2-(propan-2-yl)-4,5,6,7-tetrahydro-1H-1,3-benzodiazole instead of PF-74's methylindole group, was chosen for synthesis and biological testing. To allow the quick analysis of properties, before isolation of enantiomers, CX03 was synthesized as a racemic mixture. The direct binding to hexameric HIV-1_{NL4-3} CA, antiviral potency, and the metabolic stability of CX03 was tested and compared to PF-74 (FIGS. 5 and 6). Unfortunately, CX03 displayed a 10-fold reduction in potency, while only a 4-fold improvement in metabolic stability (FIG. 6).

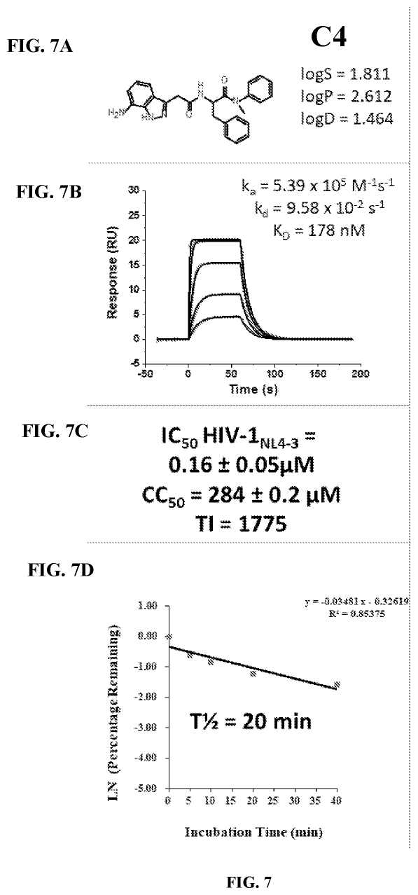

Therefore, the design workflow was further optimized to aid in the design of more potent and metabolically stable compounds. As such, the results from the Spark bioisosteric replacement experiment were imported into SeeSAR (BioSolveIT Gmbh, Germany) and assessed for predicted affinity using the HYdrogen bond and DEhydration (HYDE) energy scoring function. Compounds with higher predicted affinity were then imported into StarDrop (Optibrium Ltd., UK) and assessed for ADME/PK properties, such as log S and log P, using the oral non-CNS drug scoring profile, and for metabolic vulnerability to the major CYP enzymes using the P450 module (Opribrium, UK). Finally, compounds with better drug-like metrics and metabolic stability than PF-74 as assessed using StarDrop were taken and re-docked to the CA hexamer using AutoDock Vina and Flare ((Cresset*, Litlington, Cambridgeshire, UK; www dot cresset-group dot com/flare/). From the final list of compounds that successfully passed through the above workflow, a compound in which a 1H-indazol-7-amine group replaced the methylindole headgroup of PF-74 (designated C4) was synthesized (as a racemate) and tested. As can be seen in FIG. 7, C4 interacts with the CA hexamer similarly to PF-74, has better drug-like parameters, and has an IC₅₀ that is two-fold lower than PF-74 and over 7-fold increase in the therapeutic index (CC₅₀/IC₅₀) over PF-74. Most importantly. C4 has a 34-fold increase in metabolic stability over PF-74 as assessed using a human liver microsomal stability assay.

Compounds of Formula I such as C4 are novel drug-like compounds that interact HIV-1 CA in a functionally relevant region, and display superior properties (potency, drug-like metrics, therapeutic index, and metabolic stability) over a lead compound in the field, PF-74.

### Methods

The invention includes a method of treating, inhibiting, or suppressing an HIV-1 infection in a subject in need thereof by administering to the subject a therapeutically effective amount of at least one compound of Formula I. In some embodiments, the method comprises administering to the subject a composition comprising a therapeutically effective amount of at least one compound selected from the group consisting of:

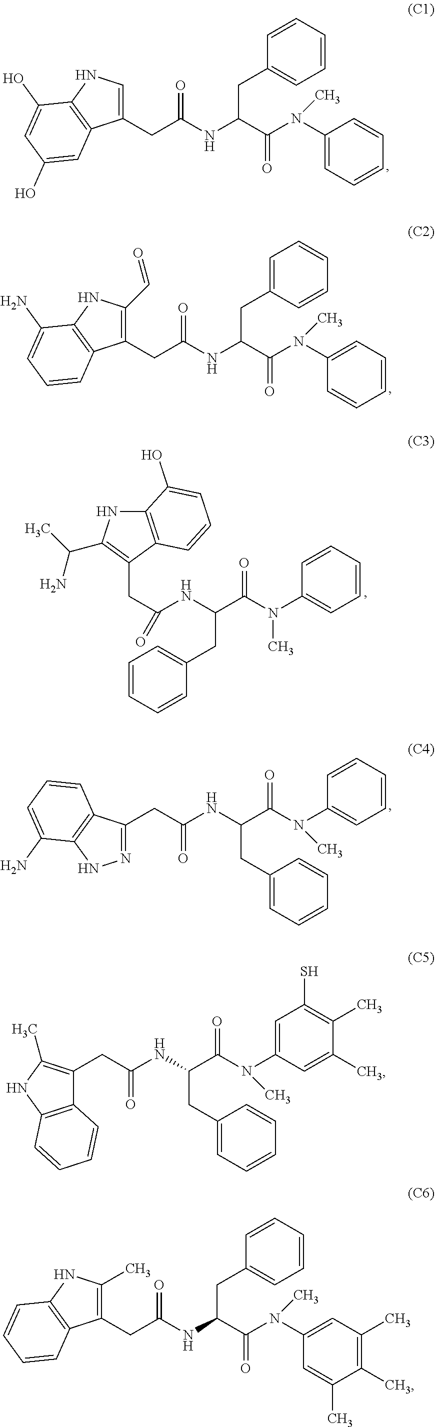

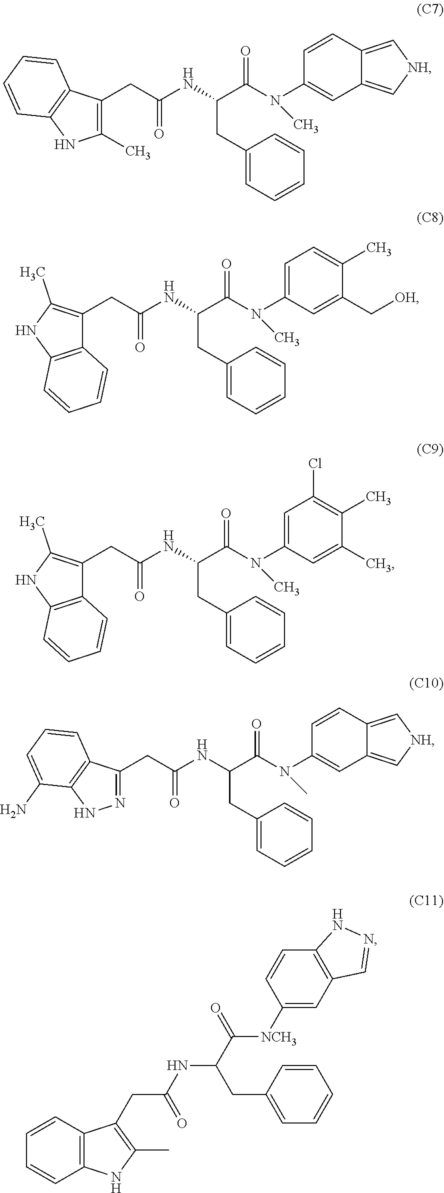 [SMILES: Cc1[nH]c2ccccc2c1CC(=O)N[C@@H](Cc1ccccc1)C(=O)N(C)c1ccc2c[nH]cc2c1 ; Cc1ccc(N(C)C(=O)[C@H](Cc2ccccc2)NC(=O)Cc2c(C)[nH]c3ccccc23)cc1CO ; Cc1cc(N(C)C(=O)[C@H](Cc2ccccc2)NC(=O)Cc2c(C)[nH]c3ccccc23)cc(Cl)c1C ; CN(C(=O)C(Cc1ccccc1)NC(=O)Cc1n[nH]c2c(N)cccc12)c1ccc2c[nH]cc2c1 ; Cc1[nH]c2ccccc2c1CC(=O)NC(Cc1ccccc1)C(=O)N(C)c1ccc2[nH]ncc2c1]

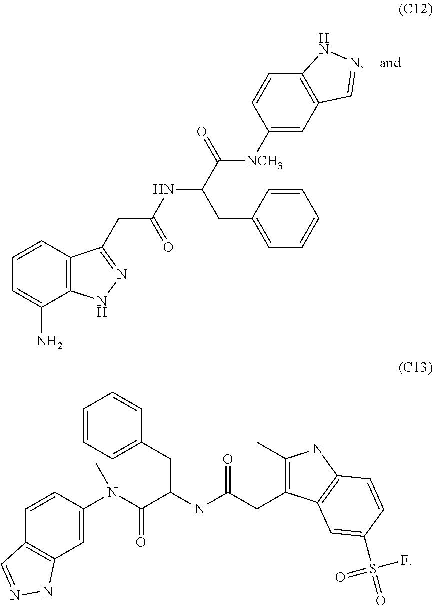 [SMILES: CN(C(=O)C(Cc1ccccc1)NC(=O)Cc1n[nH]c2c(N)cccc12)c1ccc2[nH]ncc2c1 ; Cc1[nH]c2ccc(S(=O)(=O)F)cc2c1CC(=O)NC(Cc1ccccc1)C(=O)N(C)c1ccc2cn[nH]c2c1]

In some embodiments, the method comprises administering to the subject a composition comprising a therapeutically effective amount of at least one compound selected from the group consisting of:

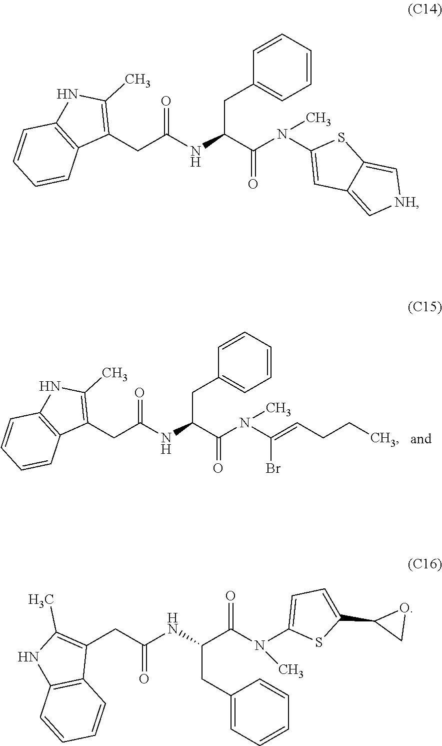 [SMILES: Cc1[nH]c2ccccc2c1CC(=O)N[C@@H](Cc1ccccc1)C(=O)N(C)c1cc2c[nH]cc2s1 ; CCC/C=C(\Br)N(C)C(=O)[C@H](Cc1ccccc1)NC(=O)Cc1c(C)[nH]c2ccccc12 ; Cc1[nH]c2ccccc2c1CC(=O)N[C@@H](Cc1ccccc1)C(=O)N(C)c1ccc([C@@H]2CO2)s1]

### Combination Therapies

The compounds identified using the methods described here are useful in the methods of the invention in combination with one or more additional compounds useful for treating HIV infections. These additional compounds may comprise compounds identified herein or compounds, e.g., commercially available compounds, known to treat, prevent, or reduce the symptoms of HIV infections.

The compounds of Formula I can be formulated in a composition that includes at least one pharmaceutically acceptable carrier, as described herein. Additionally, compositions of compounds of Formula I can further include least one additional agent that treats HIV-1 infection in a subject. Suitable additional agents include of HIV combination drugs, entry and fusion inhibitors, integrase inhibitors, non-nucleoside reverse transcriptase inhibitors, nucleoside reverse transcriptase inhibitors, and protease inhibitors.

In non-limiting examples, the compounds useful within the invention may be used in combination with one or more of the following anti-HIV drugs:

HIV Combination Drugs: efavirenz, emtricitabine or tenofovir disoproxil fumarate (Atripla®/BMS, Gilead); lamivudine or zidovudine (Combivir®/GSK): abacavir or lamivudine (Epzicom®/GSK); abacavir, lamivudine or zidovudine (Trizivir®/GSK): emtricitabine, tenofovir disoproxil fumarate (Truvada®/Gilead).
Entry and Fusion Inhibitors: maraviroc (Celsentri®, Selzentry®/Pfizer); pentafuside or enfuvirtide (Fuzeon®/Roche, Trimeris).
Integrase Inhibitors: raltegravir or MK-0518 (Isentress®/Merck).
Non-Nucleoside Reverse Transcriptase Inhibitors: delavirdine mesylate or delavirdine (Rescriptor®/Pfizer); nevirapine (Viramune®/Boehringer Ingelheim); stocrin or efavirenz (Sustiva®/BMS); etravirine (Intelence®/Tibotec).
Nucleoside Reverse Transcriptase Inhibitors: lamivudine or 3TC (Epivir®/GSK); FTC, emtricitabina or coviracil (Emtriva®/Gilead); abacavir (Ziagen®/GSK); zidovudina, ZDV, azidothymidine or AZT (Retrovir®/GSK); ddI, dideoxyinosine or didanosine (Videx®/BMS): abacavir sulfate plus lamivudine (Epzicom®/GSK); stavudine, d4T, or estavudina (Zerit®/BMS): tenofovir, PMPA prodrug, or tenofovir disoproxil fumarate (Viread®/Gilead).
Protease Inhibitors: amprenavir (Agenerase®/GSK, Vertex); atazanavir (Reyataz®/BMS); tipranavir (Aptivus®/Boehringer Ingelheim); darunavir (Prezist®/Tibotec): fosamprenavir (Telzir®, Lexiva®/GSK, Vertex); indinavir sulfate (Crixivan®/Merck): saquinavir mesylate (Invirase®/Roche); lopinavir or ritonavir (Kaletra®/Abbott); nelfinavir mesylate (Viracept®/Pfizer); ritonavir (Norvir®/Abbott).

In some embodiments, when a compound of Formula I and the least one additional agent that treats HIV-1 infection in a subject are administered together (either sequentially or concurrently), a synergistic effect is observed. A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

Routes of administration of any of the compositions of the invention include oral, nasal, rectal, intravaginal, parenteral, buccal, sublingual or topical.

The regimen of administration may affect what constitutes an effective amount. The therapeutic formulations may be administered to the subject either prior to or after the onset of a viral infection. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to treat a viral infection in the subject. An effective amount of the therapeutic compound necessary to achieve a therapeutic effect may vary according to factors such as the state of the disease or disorder in the subject: the age, sex, and weight of the subject; and the ability of the therapeutic compound to treat a viral infection in the subject. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound useful within the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

In particular, the selected dosage level will depend upon a variety of factors, including the activity of the particular compound employed, the time of administration, the rate of excretion of the compound, the duration of the treatment, other drugs, compounds or materials used in combination with the compound, the age, sex, weight, condition, general health and prior medical history of the subject being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds useful within the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on the unique characteristics of the therapeutic compound and the particular therapeutic effect to be achieved, and the limitations inherent in the art of compounding/formulating such a therapeutic compound for the treatment of an HIV-1 infection in a subject.

In one embodiment, the compositions of the invention are formulated using one or more pharmaceutically acceptable excipients or carriers. In one embodiment, the pharmaceutical compositions of the invention comprise a therapeutically effective amount of a compound useful within the invention and a pharmaceutically acceptable carrier.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from individual to individual depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds useful within the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound useful within the invention is from about 1 mg and about 2,500 mg. In some embodiments, a dose of a compound useful within the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., an HIV-1 antiviral) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments therebetween.

In one embodiment, the present invention is directed to a packaged pharmaceutical composition comprising a container holding a therapeutically effective amount of a compound useful within the invention, alone or in combination with a second pharmaceutical agent; and instructions for using the compound to treat, prevent, or reduce one or more symptoms of an HIV-1 infection in a subject.

The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration:

For oral administration, the compositions of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OYT ype, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration:

For parenteral administration, the compositions of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms:

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 2003/0147952, 2003/0104062, 2003/0104053, 2003/0044466, 2003/0039688, and 2002/0051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 1/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds useful within the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing:

The therapeutically effective amount or dose of a compound of the present invention will depend on the age, sex and weight of the subject, the current medical condition of the subject and the nature of the infection by an HIV-1 being treated. The skilled artisan will be able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days.

The compounds for use in the method of the invention may be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: Multi-Step Design and Synthesis of CX Compounds with Optimized Potency, Drug-Like Metrics, and Metabolic Stability

An efficient computational workflow that resulted in the unexpected discovery of compound C4 is described herein. This workflow was applied to different regions of the parental compound PF-74 and choose compounds with predicted potency, drug-like metrics, and metabolic stability that are superior to PF-74. Changes in different regions of the molecule that improve potency and other drug-like properties are combined to make chimeric compounds that have better properties than their precursor compounds. If chiral starting materials are available, each single enantiomer was synthesized to assess stereotopic binding. Otherwise, racemic compounds are made and evaluated, and enantiomers isolated after demonstration of antiviral activity and target interaction. In addition to the computational strategy outlined above, we also apply traditional drug design based upon iterative analoging around PF-74 and directed by the available crystal structures of the compound complexed with the CA hexamer. The findings from both the computational and traditional optimization strategies will be used together to make additional compounds.

Compounds C4, C11, and C12 were synthesized according to Schemes 1, 1, and 2 below, respectively.

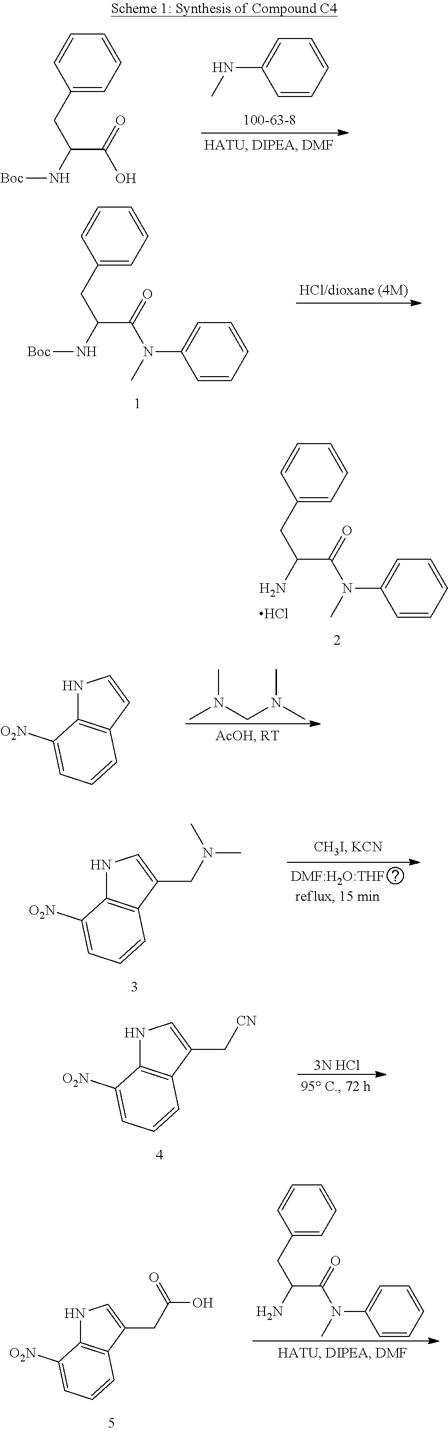

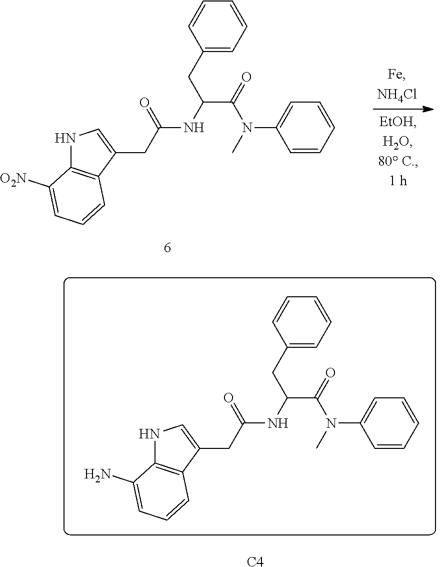 [SMILES: CN(C(=O)C(Cc1ccccc1)NC(=O)Cc1c[nH]c2c([N+](=O)[O-])cccc12)c1ccccc1 ; CN(C(=O)C(Cc1ccccc1)NC(=O)Cc1c[nH]c2c(N)cccc12)c1ccccc1 ; "C4"]

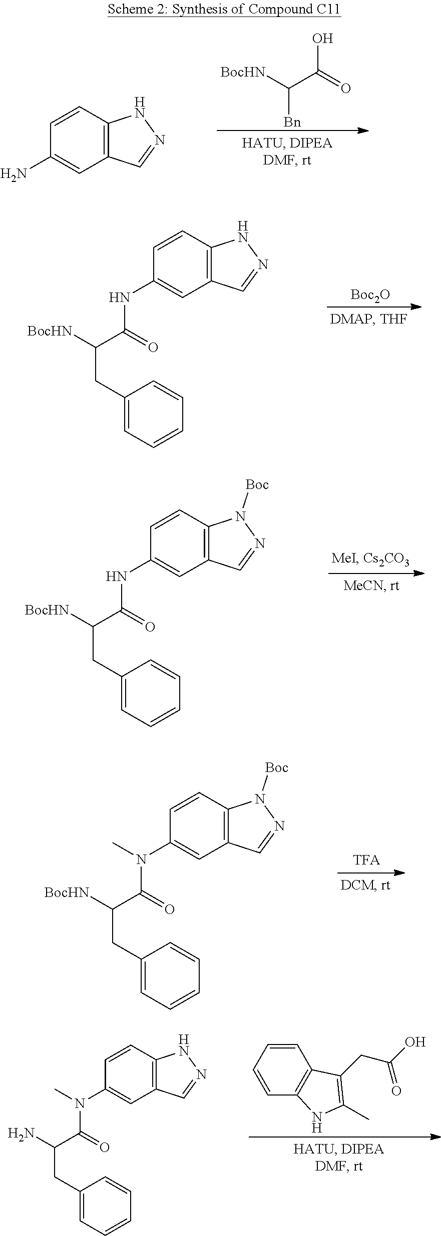

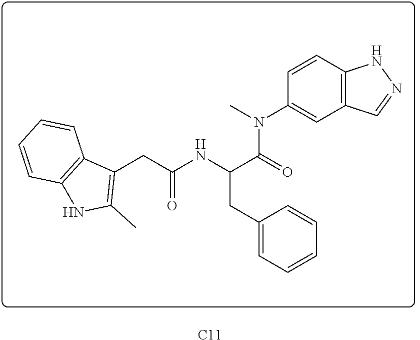 [SMILES: Cc1[nH]c2ccccc2c1CC(=O)NC(Cc1ccccc1)C(=O)N(C)c1ccc2[nH]ncc2c1 ; "C11"]

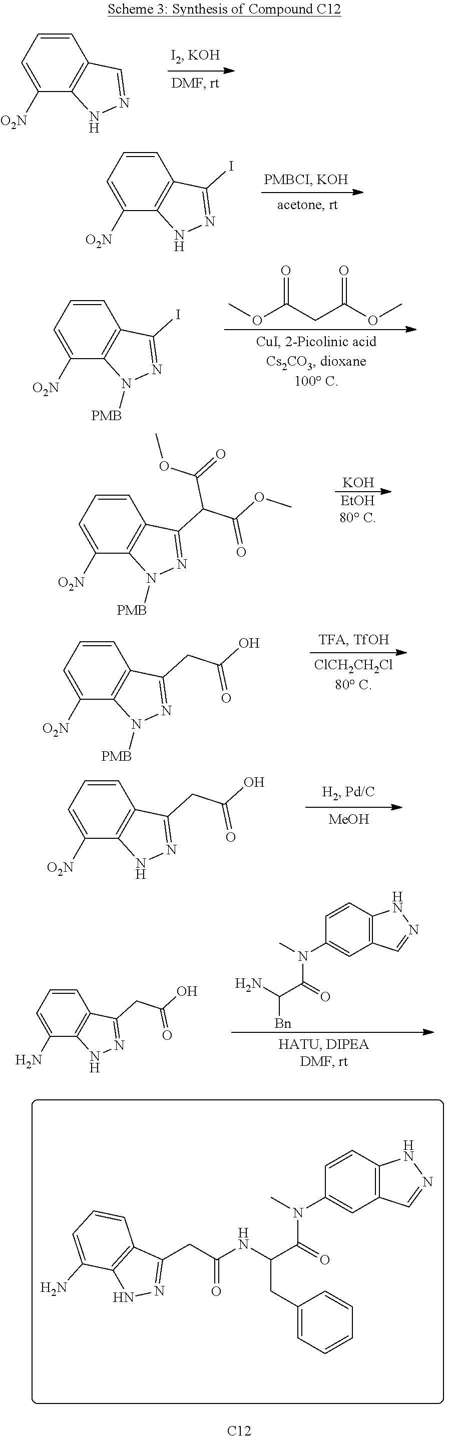

### Example 2: Target Validation, Potency and Toxicity Testing of Next-Generation CX Compounds

Compounds designed and synthesized in Example 1 are subject to a screening and validation cascade. This cascade includes analysis of direct binding of compounds to CA proteins from HIV-1 from clades A1, A2, B, C, and D using surface plasmon resonance (SPR). Also, activity and toxicity profiling of the compounds are performed in a single round infection assay using cell lines and using a multicycle HIV-1 replication assay utilizing primary isolates representing the major clades (A1, A2, B, C, and D) and primary human peripheral blood mononuclear cells. Finally, an SPR-based competition assay with peptides derived from CPSF6 and NUP153 is used to confirm that the compounds interact in the functionally relevant inter-protomer pocket, before investigating the fine mapping of contact residues using site-directed mutagenesis. This allows identification of compounds that have superior potency and toxicity profiles over the parental compound.

### Example 3

Multi-Step Design and Synthesis of Compounds of Formula I with Optimized Potency, Drug-Like Metrics, and Metabolic Stability.

### Computational Design of Compounds.

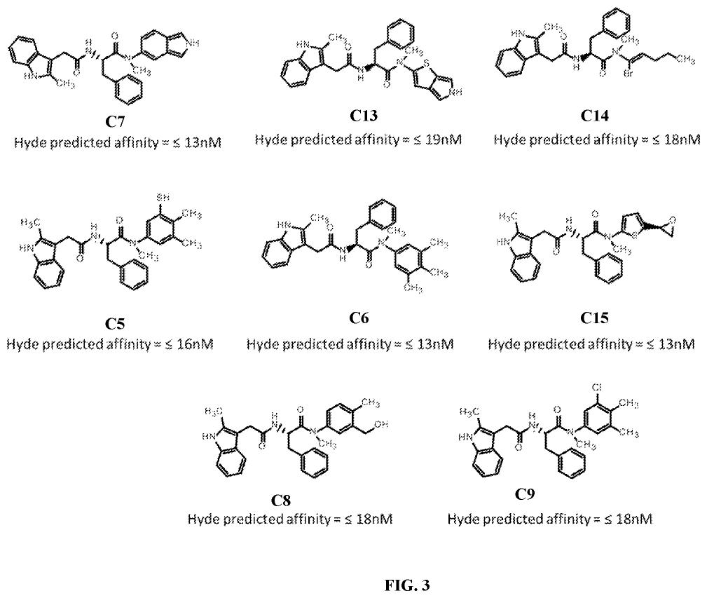

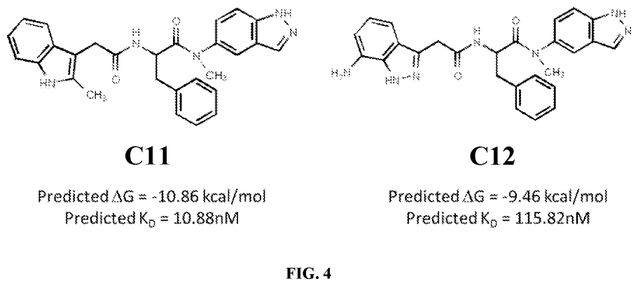

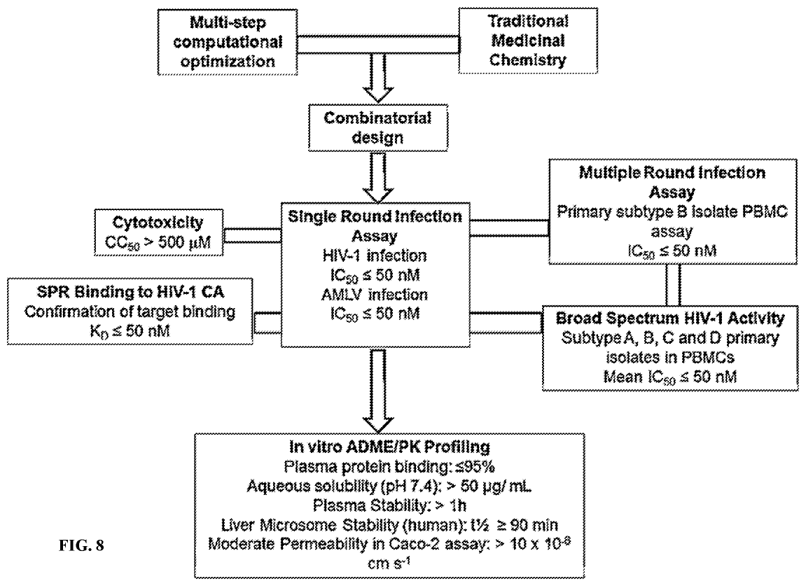

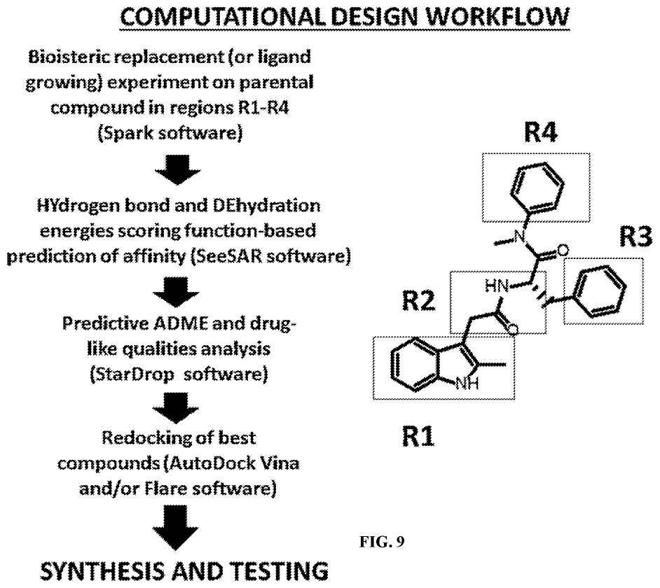

The computational workflows described herein were applied to different regions of the starting compound PF-74 (FIG. 9) and compounds were chosen with predicted potency, drug-like metrics, and metabolic stability that are superior to PF-74. This methodology was applied to the redesign of the compound by changing regions R1 and R4 of PF-74 and have identified 12 compounds with better predicted properties than PF-74 and predicted affinities of less than 20 nM (FIG. 3). These compounds were synthesized and subject to the screening cascade outlined in FIG. 8. Changes in different regions of the molecule that improve potency and other drug-like properties are combined to make chimeric compounds that can have even better properties than their precursor compounds. Initially, the compounds are synthesized as racemic mixtures to facilitate preliminary activity testing; then once the activity is confirmed, the enantiomeric forms are separated (or synthesized if chiral materials are available) and subjected to biological analysis to identify the most potent enantiomer or diastereomer.

Exploratory Medicinal Chemistry.

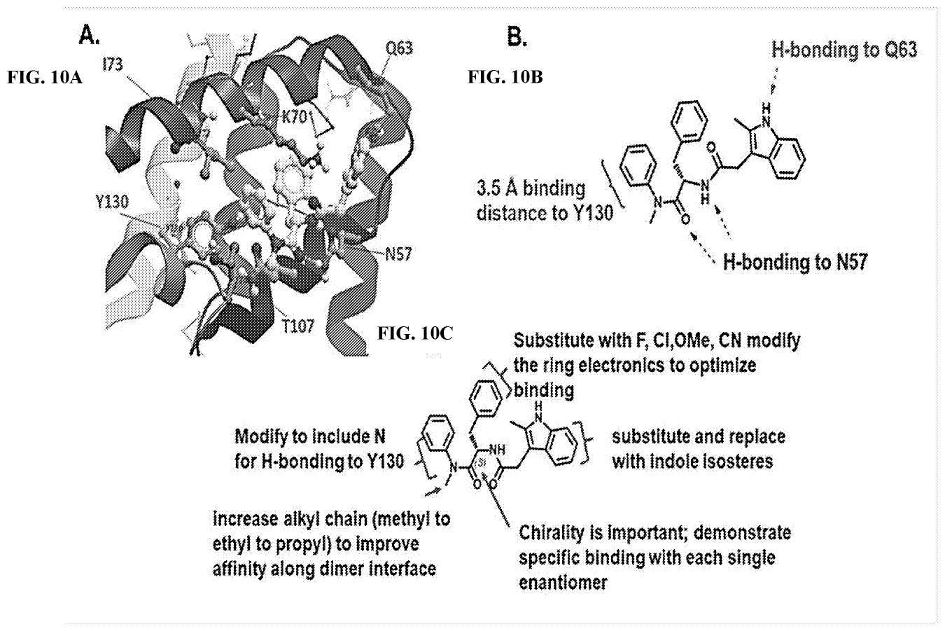

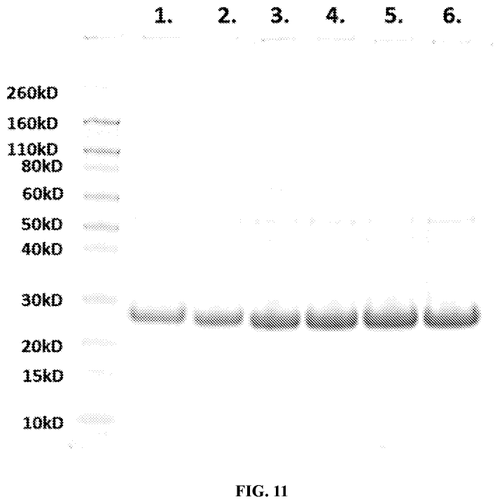

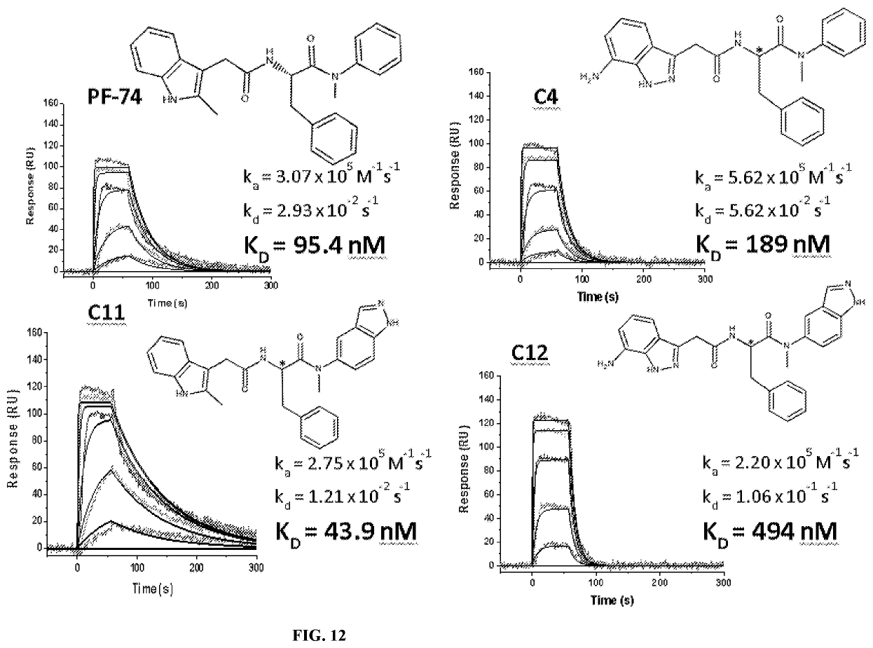

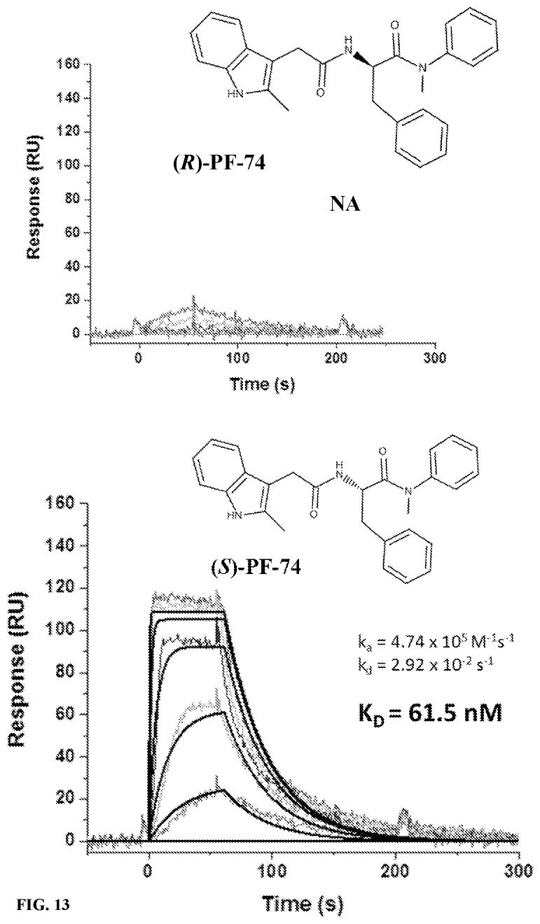

In addition to the computational strategy outlined above, traditional drug design approaches based upon iterative analoging around PF-74 and directed by the available crystal structures of the compound complexed with the CA hexamer. In FIG. 10, the X-ray structure of PF-74 in complex with the HIV-1 CA hexamer is shown. The aniline aromatic group is in close proximity to Y130, thus it is expected that a pyridine replacement for the phenyl ring will increase binding affinity. The N-methyl sits at the groove formed at the interface of two protomer units and increasing alkyl chain length from methyl to ethyl to propyl is expected to improve Van der Waals interactions and increase binding affinity. There are key H-bonds to the amide carbonyl and NH as well as to the NH of the indole N. Chirality is important for binding, and as such potency (˜10-fold difference in affinity between the eutomer and distomer), and we therefore plan to synthesize several pairs of single enantiomers to demonstrate binding in the stereotopic environment of the inter-protomer pocket. The phenyl alanine aromatic is sitting in a lipophilic aromatic pocket, and optimizing the electronics on the ring through substitution with F, Cl, OMe, or CN will modulate binding affinity. The indole may be replaced by indazine or aza-indole and other similar aromatic groups (i.e., naphthalene). This group is also at the dimer interface between two protomer units and substitution with electron donating or electron withdrawing substituents optimize binding affinity. All new analogs are evaluated for binding affinity and kinetics using SPR analysis and first-line antiviral activity using the single-round infection assay before subsequently being assessed in other assays of the critical path flow chart.

### Example 4: Target Validation, Potency and Toxicity Testing of Next-Generation CX Compounds

### Direct Binding of Compounds Hexameric HIV-1 CA Via SPR

A direct binding assay was established using SPR (surface plasmon resonance) (FIGS. 5-7). This assay allows for the confirmation that maintained target specificity is maintained, and the analysis of the kinetic signatures of newly synthesized compounds allows for prioritization of compounds for antiviral testing. Comparing the relatively low-affinity CX03 compound's kinetics to those of the higher affinity PF-74 and C4 compounds (FIGS. 5-7), it is clear that the difference primarily resides in the off-rate. Therefore, compounds with slower off-rates are prioritized for antiviral testing, as this characteristic should translate to increased potency. Additionally, the interaction of the next-generation compounds with CA proteins from clades A1, A2, B, C, and D (FIG. 11) was quantified. Hexameric versions of these constructs were developed, given their differential assembly properties, compared to the NL4-3 variant. This can be accomplished by modelling of the CA variants and mutation of residues (either A14C/E45C or A42C/T54C) between the N-terminal domains of adjacent subunits. Second, the cross-linked hexamers were prevented from polymerizing further into hyperstable capsid-like structures by mutations (W184A and M185A). The application of this to make hexameric capsomrs was first outlined in Pomillos et al. 2010. J. Mol. Biol. 401(5):985-95. The SPR analysis indicates a compound's potential performance in the therapeutic spectrum testing against other clades in the following sub-aim and provide a facile way in which to rank order compounds for antiviral testing.

### Potency and Toxicity Analysis of Next-Generation Compounds

The potency of compounds of Formula I was evaluated via single round and multicycle HIV-1 replication assays. The single round assay is used as the first level of potency testing. Compounds that display antiviral activity equal to or greater than PF-74 in the primary assay are quantified in the multicycle HIV-1 assays. The multicycle HIV-1 replication assay uses primary isolates and primary human peripheral blood mononuclear cells (PBMCs). Toxicity is evaluated simultaneously with the cells utilized in each assay to allow determination of the therapeutic index (TI=CC₅₀/IC₅₀) of each compound. HIV-1 replication assays are performed in primary PBMCs such as described in Kortager, et al., 2012, J Virol. 86(16):8472-8481, Zentner, et al., 2013, ChemMedChem. 8(3):426-432 and Zentner, et al., 2013, Bioorg Med Chem Lett. 2013; 23(4): 1132-1135.

The compounds were also assessed in human liver microsomal stability assays, plasma protein binding, aqueous solubility (pH 7.4), and evaluation of permeability using the Caco-2 assay.

Competition Experiments with CPSF6 and NUP153.

The targeted inter-protomer pocket is also the binding site for the host cellular proteins CPSF6 and NUP153. Therefore, a facile way in which to determine whether this region is targeted is to assess whether or not the compounds designed and synthesized in this study compete with peptides derived from CPSF6 or NUP153. We have already demonstrated our ability to perform an SPR-based competition assay as described in Xu, et al., 2016, Bioorg Med Chem Lett. 26(3):824-828. Briefly, we synthesized two peptides CPSF6₃₀₈₋₃₂₇ (SEQ ID NO: 1, Biot-DRPPPPVLFPGQPFGQPPLG) and a scrambled version of this peptide (SEQ ID NO: 2, PPGLVQDGFPFPQPGPPPRL), both biotinylated at the N-terminus to allow capture on a streptavidin-coated sensor chip. We also synthesized a non-biotinylated version of the CPSF6 peptide (CPSF6₃₁₃₋₃₂₇) as a control (SEQ ID NO: 3, DRPPPPVLFPGQPFGQPPLG). Both CPSF6 peptides have been previously demonstrated to interact with hexameric CA. CA hexamer at a concentration of 5 μM, either alone or in combination with high concentrations of the non-biotinylated peptide, compounds from this study or PF-74, as an additional control, are then passed over the experimental and control surface, and the response recorded. The degree of inhibition is established by comparison to the experiment performed without compounds.

### Binding Site Prediction Testing.

The computational workflow described herein includes the generation of binding site hypotheses using various docking software suites. The plausibility of the binding poses was judged in light of the experimental data, and the most plausible chosen and tested using a combination of site-directed mutagenesis of our CA constructs and interaction analysis using SPR. Mutant HIV-1_{NL4-3} CA proteins with changes in the inter-protomer pocket in both the monomeric and hexameric configurations. Additional mutants are made as required, based upon the binding site hypotheses generated.

### Evaluation in the Preliminary In Vitro ADME/PK Properties of Compounds of Formula I.

One important consideration in the redesign of a compound in this inhibitor class is to derive compounds with improved ADME/PK properties. Analysis in silico of the drug-like properties of the compounds generated in this study is used to drive compound selection. Physicochemical properties (log S, log P, etc.) of all of the new scaffolds are evaluated and compared to PF-74 using StarDrop (Optibrium, UK). The likelihood of metabolism of our new scaffolds, as compared to PF-74, by the common CYP P450 isoforms, was assessed using StarDrop and its P450 module (www dot optibrium dot com/stardrop/stardrop-p450-models dot php). StarDrop's P450 models use quantum mechanical calculations of the activation energy for hydrogen abstraction or direct oxidation, based on the semi-empirical AMi method. These are combined with ligand-based models of the steric and orientation effects of the binding pockets of each P450 isoform on the accessibility of each potential site of metabolism. This total analysis results in predictions of the regioselectivity of metabolism and the lability of each site, a measure of the efficiency of product formation—both important factors governing the rate of metabolism. Compounds of Formula I identified herein that meet the criteria for potency and cytotoxicity are evaluated in in vitro ADME assays (FIG. 8). Specifically, we use microsomal stability and plasma protein binding assessment as reliable indicators of clearance and half-life in vivo. Aqueous solubility is evaluated because low solubility or stability in assay media is detrimental to the development of sound structure-activity-relationships. Low solubility can also hamper our ability to formulate analogs for future in vivo studies. A Caco-2 assay is used to assess cell permeability issues. Compounds that move forward can satisfy at least one of the following ADME criteria: log P 1-4; plasma protein binding<95%; plasma stability>1 hr; human microsomal t½>90 min; solubility>50 μg/mL; Caco-2 Pₐₚₚ (A−B)>1×10⁻⁶ cm s⁻¹.

Table 2 lists selected single-change compounds of Formula I, C4 and C11, which displayed improved metabolic stability over PF-74, 30- and 34-fold increases, respectively. However, the dual-change chimeric compound C12 had a 118-fold improvement in metabolic stability over PF-74, as shown in Table 3.

TABLE 2
IC₅₀ values for compounds C4, C11, and C12 for inhibition
of HIV-1 in the early stages of replication

| Compound | IC₅₀ (nM) | CC₅₀ (μM) | Therapeutic index |
|---|---|---|---|
| PF-74 | 95.1 ± 31.7 | 73 ± 15 | 768 |
| C4 | 51.5 ± 18.4 | 284 ± 0.2 | 5515 |
| C11 | 173.3 ± 58 | 366 ± 29 | 2115 |
| C12 | 302.2 ± 167 | 183 ± 15 | 606 |

TABLE 3
Comparative Metabolic stability of Compounds C4, C11, and C12

| | | T_{1/2} | CLᵢₙₜ | CLₐₚₚ | CLₕ | |
|---|---|---|---|---|---|---|
| Compound ID | k | (min) | (mL/min/mg) | (mL/min/kg) | (mL/min/kg) | Eₕ |
| Testosterone | 0.03852 | 18.0 | 0.0770 | 74.292 | 15.758 | 78.79 |
| Propranolol | 0.01887 | 36.7 | 0.0377 | 36.391 | 12.907 | 64.53 |
| Warfarin | −0.00509 | stable | / | / | / | / |
| PF-74 | 1.16637 | 0.6 | 2.3327 | 2249.427 | 19.824 | 99.12 |
| C4 | 0.03481 | 20 | 0.0696 | 67.126 | 15.409 | 77 |
| C11 | 0.03934 | 18 | 0.0787 | 75.866 | 15.828 | 79.1 |
| C12 | 0.00976 | 71 | 0.0195 | 18.819 | 9.696 | 48.5 |

T_{1/2} is the liver microsomal half-life, CLᵢₙₜ, CLₐₚₚ, and CLₕ are pharmacokinetic clearance parameters.

### Example 5: Binding and Metabolic Stability of Compounds of Formula Ia and Formula Ib

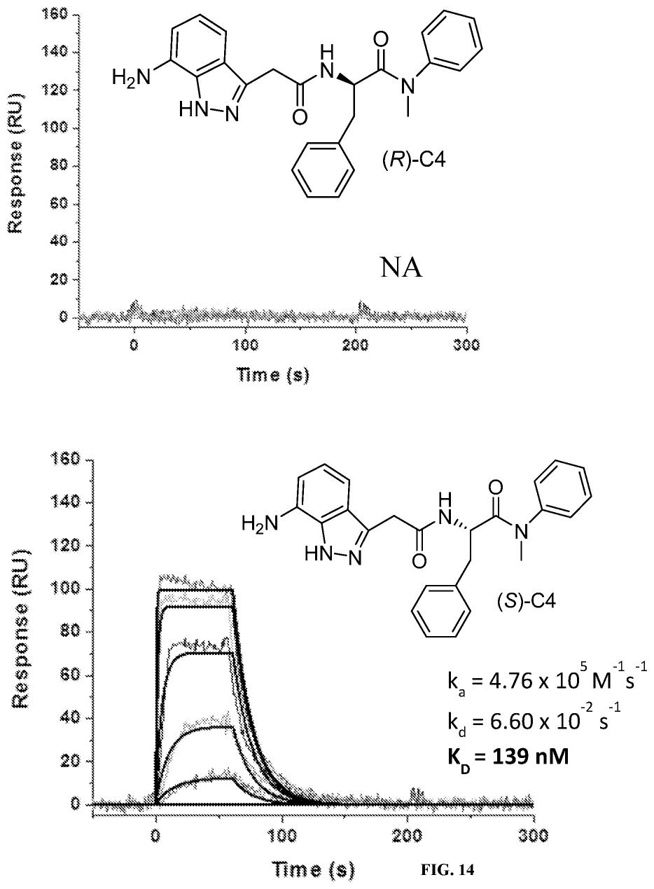

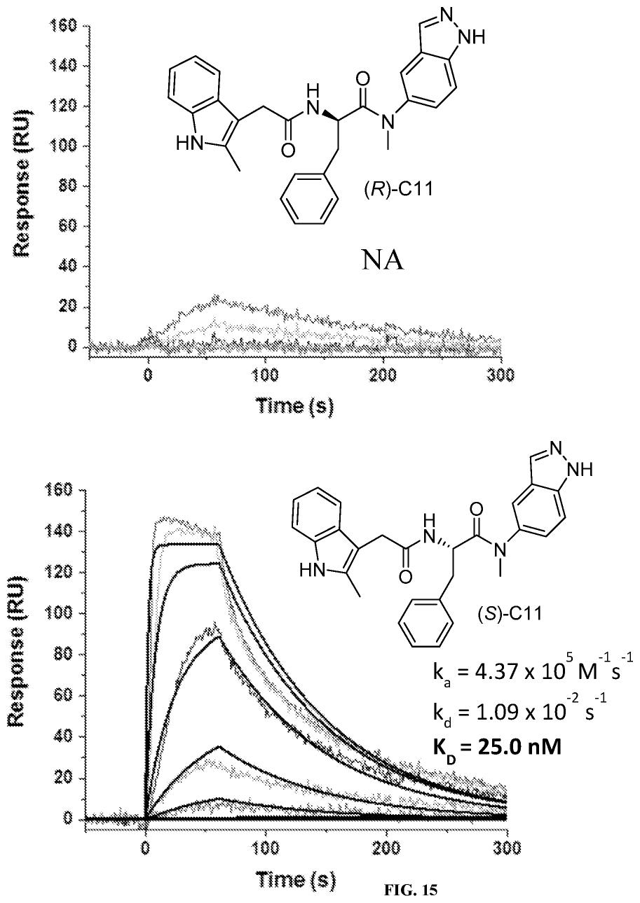

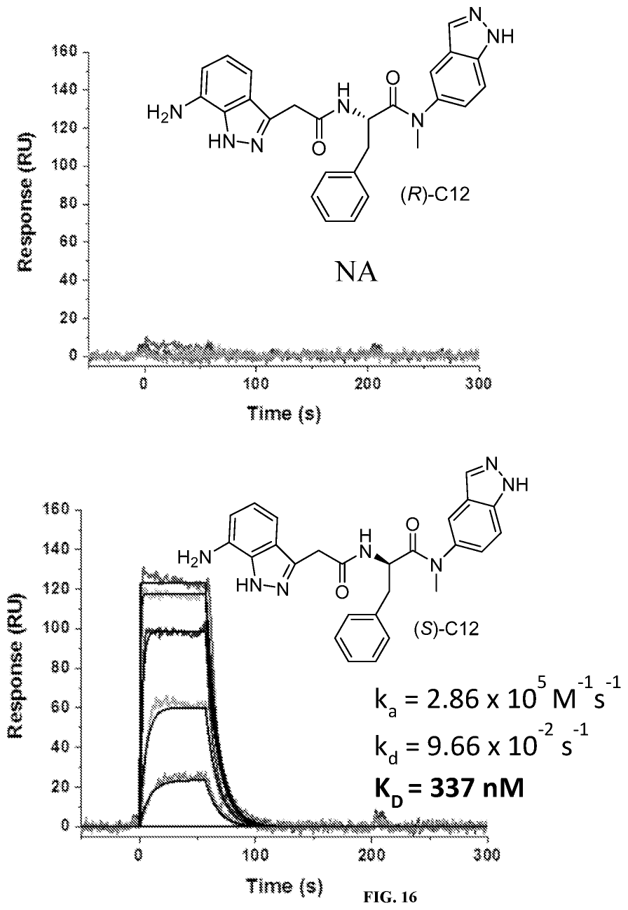

Unexpectedly in some embodiments, compound of Formula Ia bind to HIV-1 CA hexamer, whereas compounds of Formula Ib do not bind to HIV-I CA hexamer. The ability of the compound of Formula Ia to selective bind to HIV-1 CA hexamer as compared to compounds of Formula Ib is shown in FIGS. 14-16. The binding properties of both (R) and (S) C4, C11, and C12 (along with PF-74 control) is show in Table 4. The corresponding stability data for (R) and (S) C4, C11, and C12 (along with PF-74 control) is show in Table 5.

TABLE 4
Comparison of binding of compounds of Formula Ia and Ib

| | | |
|---|---|---|
| | | Therapeutic index |
| Compound | IC₅₀ | (CC₅₀/IC₅₀) |

| | | | |
|---|---|---|---|
| PF-74 | 95.1 ± 31.7 | nM | 768 |

| | | |
|---|---|---|
| (R)-PF-74 | — | — |

| | | | |
|---|---|---|---|
| (S)-PF-74 | 80 ± 5.7 | nM | 3075 |
| C4 | 51.5 ± 18.4 | nM | 5515 |

| | | |
|---|---|---|
| (R)-C4 | — | — |

| | | | |
|---|---|---|---|
| (S)-C4 | 62.1 ± 8.16 | nM | 1626 |
| C11 | 173.3 ± 58 | nM | 2115 |

| | | |
|---|---|---|
| (R)-C11 | — | — |

| | | | |
|---|---|---|---|
| (S)-C11 | 20.6 ± 1.84 | nM | 1252 |
| C12 | 302.2 ± 167 | nM | 606 |

| | | |
|---|---|---|
| (R)-C12 | — | — |

| | | | |
|---|---|---|---|
| (S)-C12 | 279 ± 52.6 | nM | 366 |

TABLE 5
Metabolic stability of compounds of Formula Ia and Ib

| Compound | T_{1/2} (min) |
|---|---|
| PF-74 | 0.6 |
| (R)-PF-74 | 0.6 |
| (S)-PF-74 | 0.5 |
| C4 | 20 |
| (R)-C4 | 18.2 |
| (S)-C4 | 15.4 |
| C11 | 17.6 |
| (R)-C11 | 20.8 |
| (S)-C11 | 1.0 |
| C12 | 71.0 |
| (R)-C12 | 48.9 |
| (S)-C12 | 102.0 |

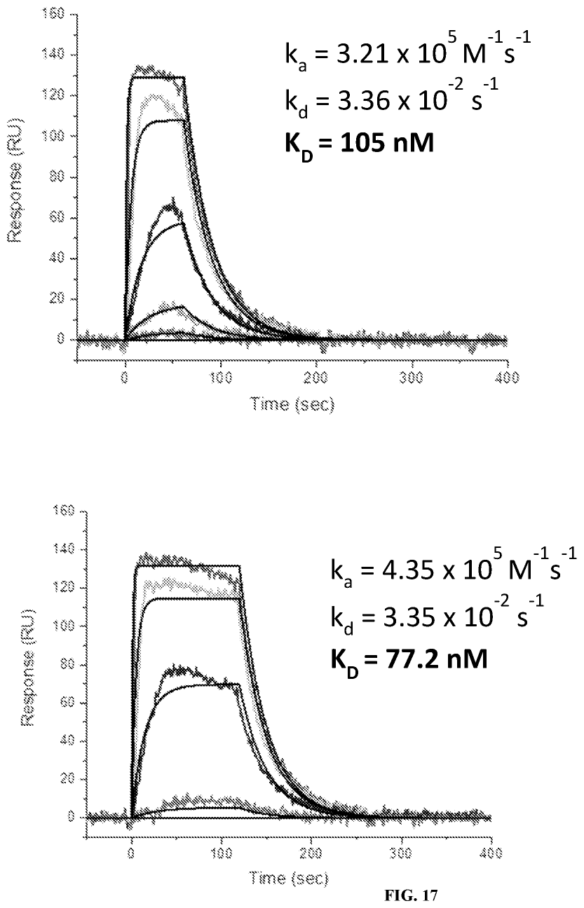

Compound C13 showed even more potent binding than (S)-C11 (FIG. 17), as show in Table 6. Compound C13, with the SO₂F reactive group, displays stable inhibition over various time point, as compared to the parental, non-SO₂F version (S)-C11, whose IC50 reduces over time. This could be indicative of a covalent interaction occurring between C13 and the HIV-1 CA.

TABLE 6
Comparison of IC50 values between C13
and (S)-C11 at different time points.

| | 24 h post- | 48 h post- | 72 h post- |
|---|---|---|---|
| Compound | infection IC₅₀ | infection IC₅₀ | infection IC₅₀ |
| C13 | 23.1 ± 4.29 nM | 23.9 ± 3.24 nM | 29.1 ± 5.67 nM |
| (S)-C11 | 33.0 ± 3.50 nM | 43.9 ± 6.34 nM | 61.4 ± 3.07 nM |

### Enumerated Embodiments

The following enumerated embodiments are provided, the numbering of which is not to be construed as designating levels of importance:

Embodiment 1 provides a compound of Formula I, or a pharmaceutically acceptable salt, solvate, or tautomer thereof:

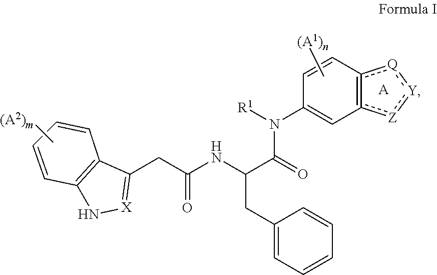

wherein

[symbol] is a covalent double bond or a covalent single bond;

ring A is optionally absent;

R¹ is hydrogen or a C₁₋₅ alkyl;

at each occurrence A¹ is independently selected from the group consisting of F, Cl, Br, I, OR, CN, NO₂, CF₃, OCF₃, R, N(R)₂, SR, SO₂F, SO₂R SO₂N(R)₂, SO₃R, and (CH₂)₁₋₃OR;

at each occurrence A² is independently selected from the group consisting of F, Cl, Br, I, OR, CN, NO₂, CF₃, OCF₃, R N(R)₂, SR SO₂F, SO₂R, SO₂N(R)₂, SO₃R, and (CH₂)₁₋₃OR;

X is N, C—C(═O)R, or C—R, wherein R is optionally substituted by 1 to 3 groups selected from the group consisting of NH₂ and OH;

Q, Y, and Z are each independently N, NH or CH, wherein at least one of Q, Y, and Z is N or NH;

at each occurrence R is independently hydrogen or C₁₋₄ alkyl;

m is an integer from 0 to 5; and

n is an integer from 0 to 5.

Embodiment 2 provides the compound of embodiment 1, wherein the compound has the structure of Formula Ia or Ib:

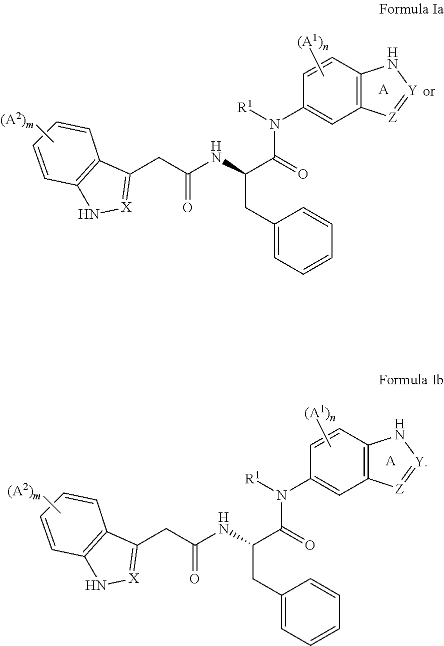

Embodiment 3 provides the compound of any one of embodiments 1-2, wherein the compound has the structure of Formula IIa or IIb:

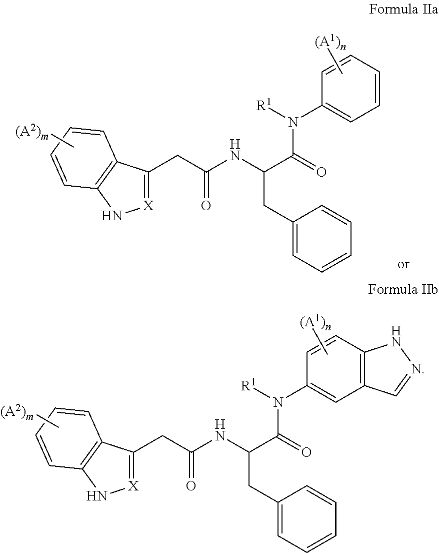

Embodiment 4 provides the compound of any one of embodiments 1-3, wherein X is N.

Embodiment 5 provides the compound of any one of embodiments 1-4, wherein X is CH or C—CH₃.

Embodiment 6 provides the compound of any one of embodiments 1-5, wherein R¹ is CH₃.

Embodiment 7 provides the compound of any one of embodiments 1-6, wherein the compound has the structure:

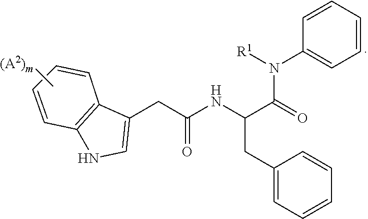

Embodiment 8 provides the compound of any one of embodiments 1-7, wherein the compound has the structure:

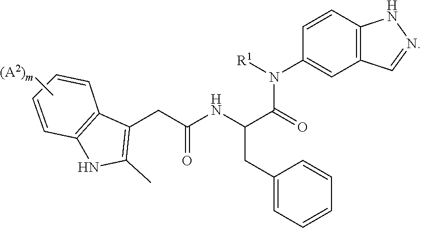

Embodiment 9 provides the compound of any one of embodiments 1-8, wherein the compound has the structure:

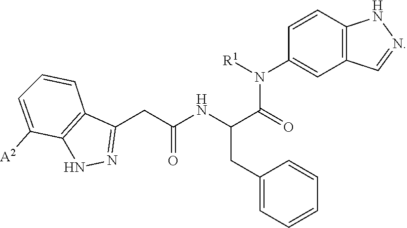

Embodiment 10 provides the compound of any one of embodiments 1-9, wherein m is 1.

Embodiment 11 provides the compound of any one of embodiments 1-10, wherein A² is NH₂.

Embodiment 12 provides the compound of any one of embodiments 1-11, wherein the compound is selected from the group consisting of:

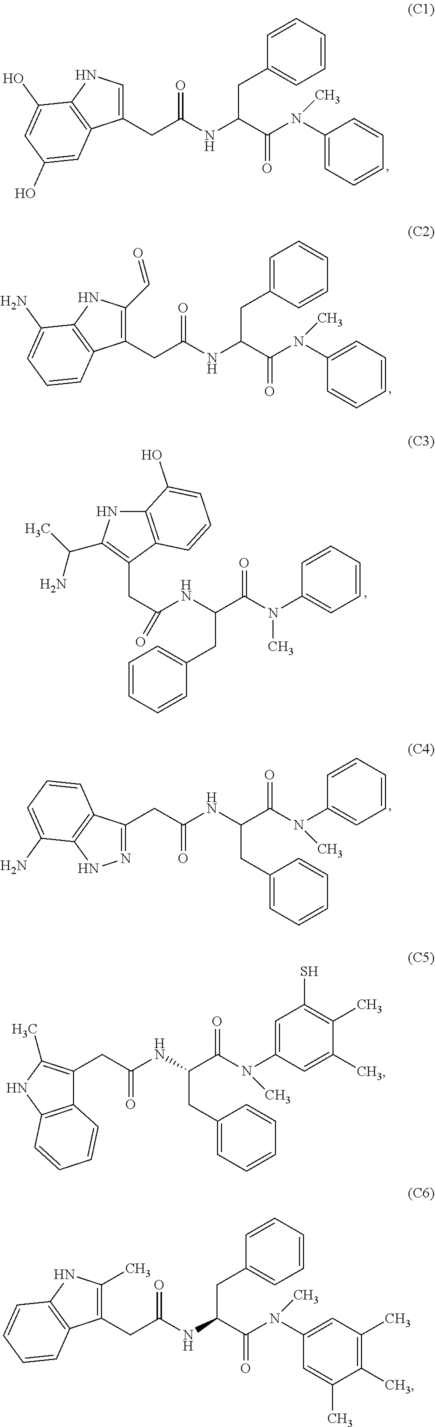

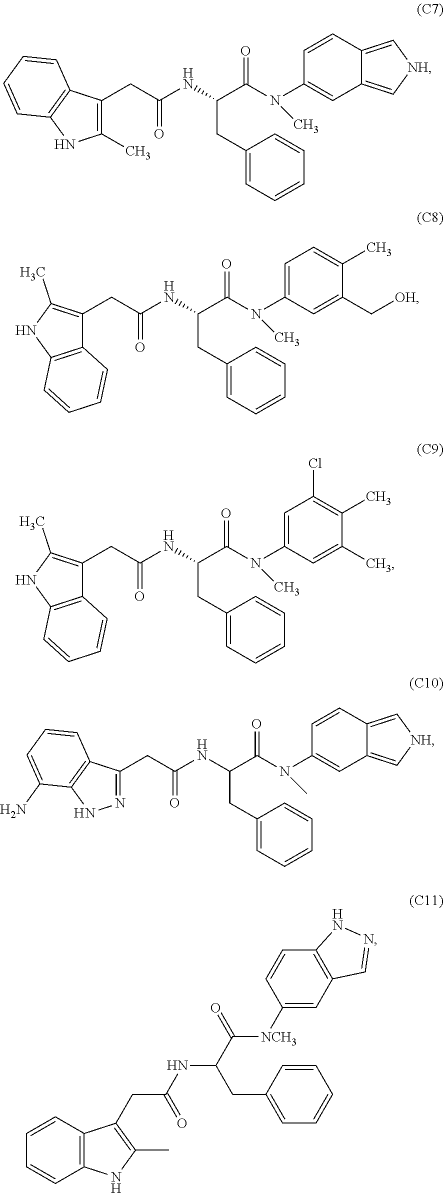 [SMILES: Cc1[nH]c2ccccc2c1CC(=O)N[C@@H](Cc1ccccc1)C(=O)N(C)c1ccc2c[nH]cc2c1 ; Cc1ccc(N(C)C(=O)[C@H](Cc2ccccc2)NC(=O)Cc2c(C)[nH]c3ccccc23)cc1CO ; Cc1cc(N(C)C(=O)[C@H](Cc2ccccc2)NC(=O)Cc2c(C)[nH]c3ccccc23)cc(Cl)c1C ; CN(C(=O)C(Cc1ccccc1)NC(=O)Cc1n[nH]c2c(N)cccc12)c1ccc2c[nH]cc2c1 ; Cc1[nH]c2ccccc2c1CC(=O)NC(Cc1ccccc1)C(=O)N(C)c1ccc2[nH]ncc2c1]

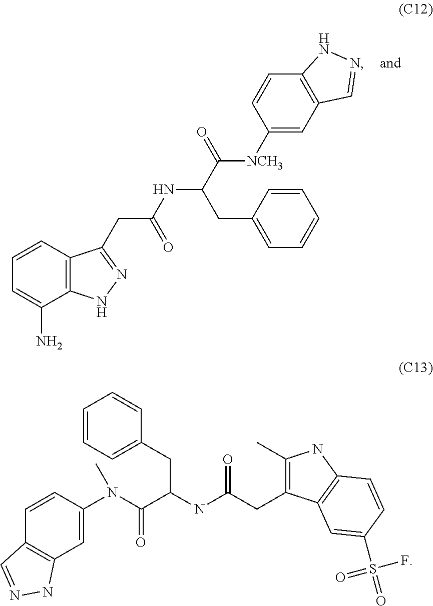 [SMILES: CN(C(=O)C(Cc1ccccc1)NC(=O)Cc1n[nH]c2c(N)cccc12)c1ccc2[nH]ncc2c1 ; Cc1[nH]c2ccc(S(=O)(=O)F)cc2c1CC(=O)NC(Cc1ccccc1)C(=O)N(C)c1ccc2cn[nH]c2c1]

Embodiment 13 provides a compound selected from the group consisting of:

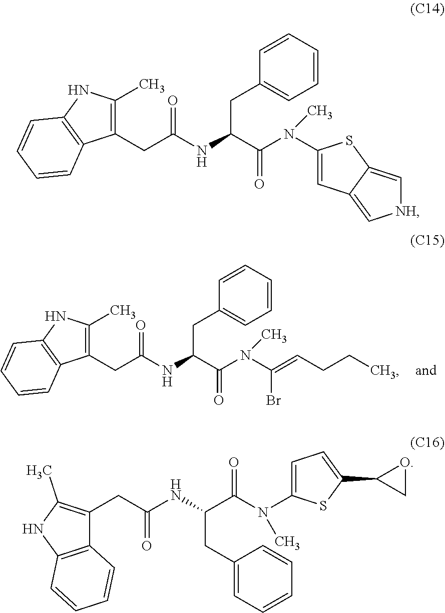 [SMILES: Cc1[nH]c2ccccc2c1CC(=O)N[C@@H](Cc1ccccc1)C(=O)N(C)c1cc2c[nH]cc2s1 ; CCC/C=C(\Br)N(C)C(=O)[C@H](Cc1ccccc1)NC(=O)Cc1c(C)[nH]c2ccccc12 ; Cc1[nH]c2ccccc2c1CC(=O)N[C@@H](Cc1ccccc1)C(=O)N(C)c1ccc([C@@H]2CO2)s1]

Embodiment 14 provides a composition comprising at least one compound of any one of embodiments 1-13 and at least one pharmaceutically acceptable carrier.

Embodiment 15 provides the composition of embodiment 14, further comprising at least one additional agent that treats HIV-1 infection in a subject.

Embodiment 16 provides the composition of any one of embodiments 14-15, wherein the at least one additional agent is selected from the group consisting of HIV combination drugs, entry and fusion inhibitors, integrase inhibitors, non-nucleoside reverse transcriptase inhibitors, nucleoside reverse transcriptase inhibitors, and protease inhibitors.

Embodiment 17 provides a method of treating, inhibiting, or suppressing an HIV-1 infection in a subject in need thereof, said method comprising administering to said subject at least one compound of any of Embodiments 1-13 or at least one compositions of any of Embodiments 14-16.

Embodiment 18 provides the method of embodiment 17, wherein the subject is further administered at least one additional agent that treats HIV-1 infection in a subject.

Embodiment 19 provides the method of any one of embodiments 17-18, wherein the at least one additional agent is selected from the group consisting of HIV combination drugs, entry and fusion inhibitors, integrase inhibitors, non-nucleoside reverse transcriptase inhibitors, nucleoside reverse transcriptase inhibitors, and protease inhibitors.

Embodiment 20 provides the method of any one of embodiments 17-19, wherein the subject is a human.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A compound of Formula I, or a pharmaceutically acceptable salt, solvate, or tautomer thereof: 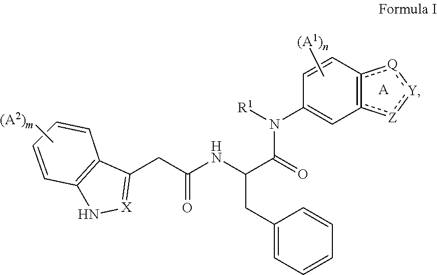
wherein [symbol]
is a covalent double bond or a covalent single bond;
ring A is optionally absent;
R¹ is hydrogen or a C₁₋₅ alkyl;
at each occurrence A¹ is independently selected from the group consisting of F, Cl, Br, I, OR, CN, NO₂, CF₃, OCF₃, R, N(R)₂, SR, SO₂F, SO₂R, SO₂N(R)₂, SO₃R, and (CH₂)₁₋₃OR;
at each occurrence A² is independently selected from the group consisting of F, Cl, Br, I, OR, CN, NO₂, CF₃, OCF₃, R, N(R)₂, SR, SO₂F, SO₂R, SO₂N(R)₂, SO₃R, and (CH₂)₁₋₃ OR;
X is N, C—C(═O)R, or C—R, wherein R is optionally substituted by 1 to 3 groups selected from the group consisting of NH₂ and OH;
Q, Y, and Z are each independently N, NH, or CH, wherein at least one of Q, Y, and Z is N or NH,
at each occurrence R is independently hydrogen or C₁₋₄ alkyl;
m is an integer from 0 to 5; and
n is an integer from 0 to 5.

2. The compound of claim 1, wherein the compound has the structure of Formula Ia or Ib:

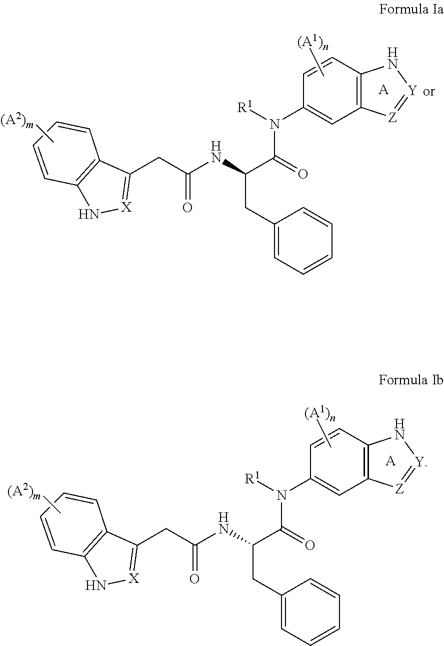

3. The compound of claim 1, wherein the compound has the structure of Formula IIa or IIb:

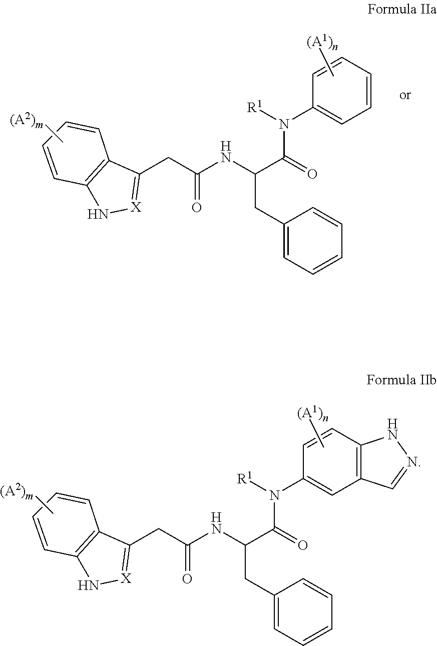

4. The compound of claim 3, wherein X is N.

5. The compound of claim 3, wherein X is CH or C—CH₃.

6. The compound of claim 1, wherein R¹ is CH₃.

7. The compound of claim 3, wherein the compound has the structure:

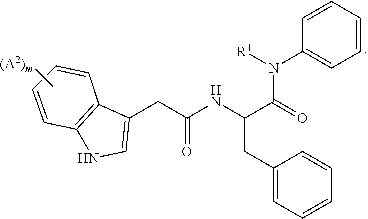

8. The compound of claim 3, wherein the compound has the structure:

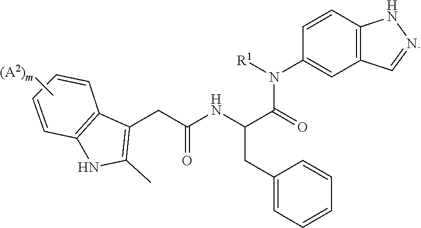

9. The compound of claim 3, wherein the compound has the structure:

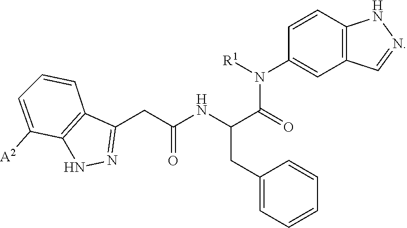

10. The compound of claim 1, wherein m is 1.

11. The compound of claim 1, wherein A² is NH₂.

12. The compound of claim 1, wherein the compound is selected from the group consisting of:

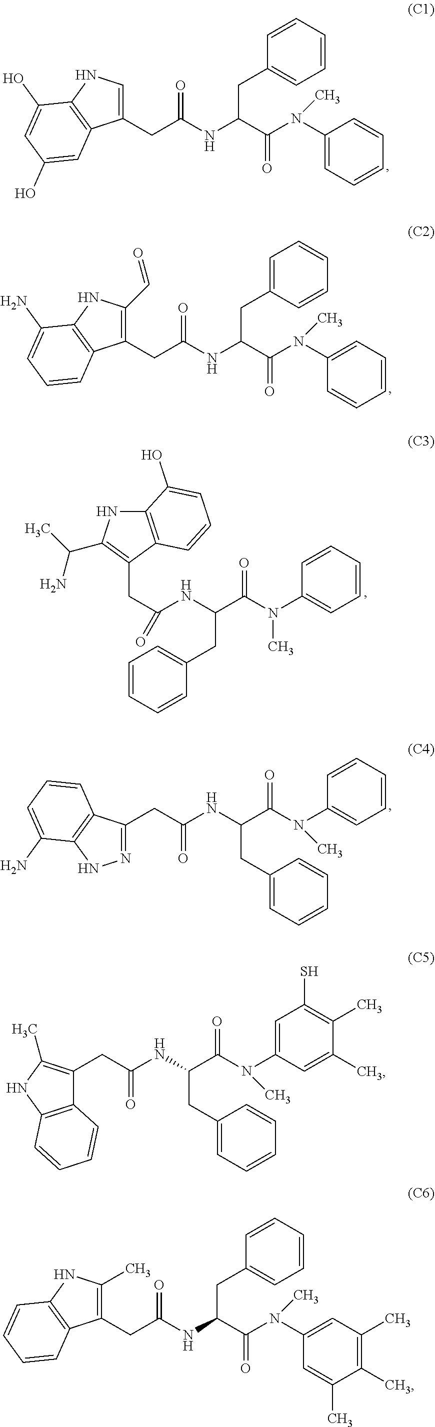

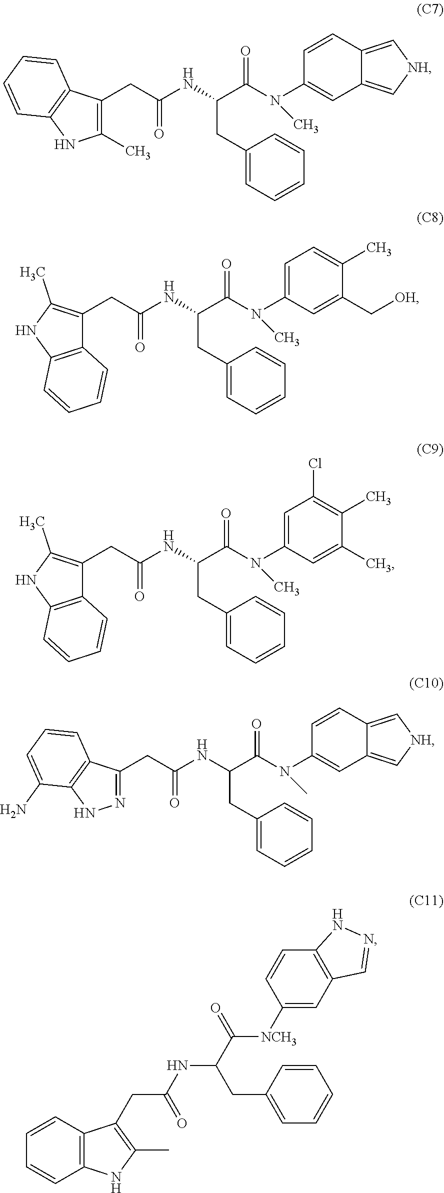 [SMILES: Cc1[nH]c2ccccc2c1CC(=O)N[C@@H](Cc1ccccc1)C(=O)N(C)c1ccc2c[nH]cc2c1 ; Cc1ccc(N(C)C(=O)[C@H](Cc2ccccc2)NC(=O)Cc2c(C)[nH]c3ccccc23)cc1CO ; Cc1cc(N(C)C(=O)[C@H](Cc2ccccc2)NC(=O)Cc2c(C)[nH]c3ccccc23)cc(Cl)c1C ; CN(C(=O)C(Cc1ccccc1)NC(=O)Cc1n[nH]c2c(N)cccc12)c1ccc2c[nH]cc2c1 ; Cc1[nH]c2ccccc2c1CC(=O)NC(Cc1ccccc1)C(=O)N(C)c1ccc2[nH]ncc2c1]

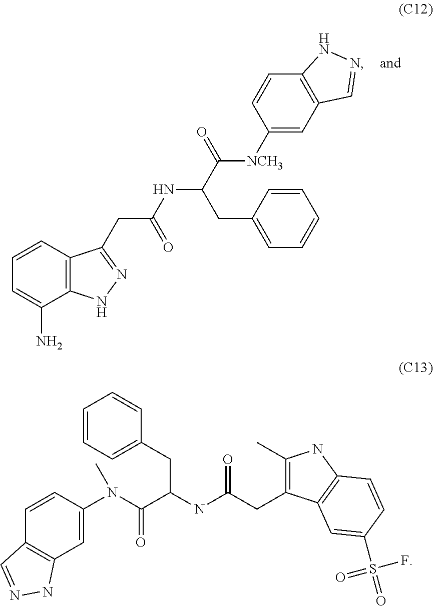 [SMILES: CN(C(=O)C(Cc1ccccc1)NC(=O)Cc1n[nH]c2c(N)cccc12)c1ccc2[nH]ncc2c1 ; Cc1[nH]c2ccc(S(=O)(=O)F)cc2c1CC(=O)NC(Cc1ccccc1)C(=O)N(C)c1ccc2cn[nH]c2c1]

13. A compound selected from the group consisting of:

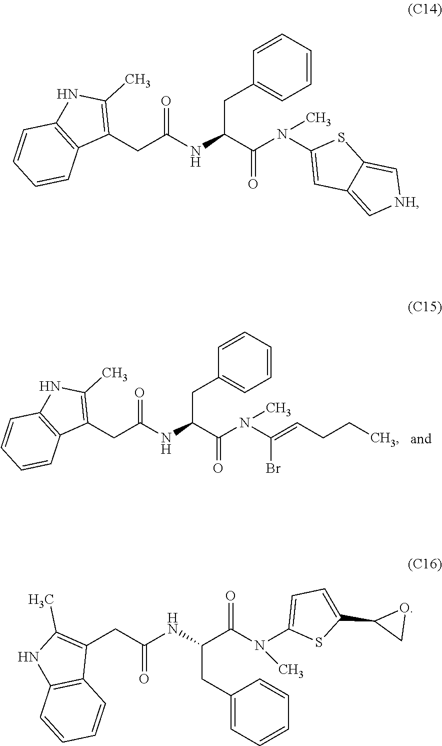 [SMILES: Cc1[nH]c2ccccc2c1CC(=O)N[C@@H](Cc1ccccc1)C(=O)N(C)c1cc2c[nH]cc2s1 ; CCC/C=C(\Br)N(C)C(=O)[C@H](Cc1ccccc1)NC(=O)Cc1c(C)[nH]c2ccccc12 ; Cc1[nH]c2ccccc2c1CC(=O)N[C@@H](Cc1ccccc1)C(=O)N(C)c1ccc([C@@H]2CO2)s1]

14. A composition comprising at least one compound of claim 1 and at least one pharmaceutically acceptable carrier.

15. The composition of claim 14, further comprising at least one additional agent that treats HIV-1 infection in a subject.

16. The composition of claim 15, wherein the at least one additional agent is selected from the group consisting of HIV combination drugs, entry and fusion inhibitors, integrase inhibitors, non-nucleoside reverse transcriptase inhibitors, nucleoside reverse transcriptase inhibitors, and protease inhibitors.

17. A method of treating, inhibiting, or suppressing an HIV-1 infection in a subject in need thereof, said method comprising administering to said subject at least one compound of claim 1.

18. The method of claim 17, wherein the subject is further administered at least one additional agent that treats HIV-1 infection in a subject.

19. The method of claim 18, wherein the at least one additional agent is selected from the group consisting of HIV combination drugs, entry and fusion inhibitors, integrase inhibitors, non-nucleoside reverse transcriptase inhibitors, nucleoside reverse transcriptase inhibitors, and protease inhibitors.

20. The method of claim 17, wherein the subject is a human.
